SEQUENCE LISTING (4 sequences)

SEQ ID NO: 1 - DNA, 1145 bp, Homo sapiens
  source 1..1145: mol_type = genomic DNA
acacccggttgggctctgctgctcccttctgggttccgaggcccaagcccttggcagtgtttgtgagtggaagggaggtcacgctatcgtccgcggccccagcagccctgtgccctcgttggatcccgcgacgcggctcctttaagagcctcgcgggtcgcccgccgctaggtcgctccccggccatgcgggcgctgcgggccggcctgaccctggcgtcgggcgcggggctgggtgcggtcgtcgagggctggcggcggcggcgggaggacgcgcgggcggcgccgggactgctgggccggctgcccgtgctgcccgtggcggcggcagccgagttgccccctgtgcccgggggaccccgcggcccgggcgagctggccaagtacgggctgccggggctggcgcagctcaagagccgcgagtcgtacgtgctgtgctacgacccgcgcacccgcggcgcgctctgggtggtggagcagctgcgacccgagcgtctccgcggcgacggcgaccggcgcgagtgcgacttccgcgaggacgactcggtgcacgcgtaccaccgtgccaccaacgccgactaccgcggcagtggcttcgaccgcggtcacctggccgccgccgccaaccaccgctggagccagaaggccatggacgacacgttctacctgagcaacgtcgcgccccaggtgccccacctcaaccagaatgcctggaacaacctggagaaatatagccgcagcttgacccgcagctaccaaaacgtctatgtctgcacagggccactcttcctgcccaggacagaggctgatgggaaatcctacgtaaagtaccaggtcatcggcaagaaccacgtggcagtgcccacacacttcttcaaggtgctgatcctggaggcagcaggtgggcaaattgagctccgcacctacgtgatgcccaacgcacctgtggatgaggccatcccactggagcgcttcctggtgcccatcgagagcattgagcgggcttcggggctgctctttgtgccaaacatcctggcgcgggcaggcagcctcaaggccatcacggcgggcagtaagtgagggtggagcccagtgagactgtgggtgtgtgcaggccggggagtattaaaggtggtgatttttgga

SEQ ID NO: 2 - protein, 297 aa, Homo sapiens
MRALRAGLTLASGAGLGAVVEGWRRRREDARAAPGLLGRLPVLPVAAAAELPPVPGGPRGPGELAKYGLPGLAQLKSRESYVLCYDPRTRGALWVVEQLRPERLRGDGDRRECDFREDDSVHAYHRATNADYRGSGFDRGHLAAAANHRWSQKAMDDTFYLSNVAPQVPHLNQNAWNNLEKYSRSLTRSYQNVYVCTGPLFLPRTEADGKSYVKYQVIGKNHVAVPTHFFKVLILEAAGGQIELRTYVMPNAPVDEAIPLERFLVPIESIERASGLLFVPNILARAGSLKAITAGSK

SEQ ID NO: 3 - DNA, 1010 bp, Caenorhabditis elegans
  source 1..1010: mol_type = genomic DNA
ccgaaatatgatcggcaaggtggctggaaccgcggcaatcgctggaatatctttcctcgctggaaaatattcgaatgacgatctgccaatcttcaggaatgttcaatcagccaccaatgttccaatgaatcaaatccaagtttcggagccaatgaccgtaaaaccagcatctctaaatgctgatgcaatgggaccatctcgttcggcagagatcatgaagcacggatatcccggattcacaaatgttcgaacttatgaggatttcgtgctttcctacgattataagacaagaactgcacactgggtctgcgaacatcttacaccagaacgattaaaacacgcagaaggtgtggatcggaagctttgtgaattcaagccagatatcactttcccgcagaaattcctttctcaaaatacggattataagtgcagtggattcgatcgtggacatctcgccgccgctggaaatcatcggaagagtcagttggcggtcgatcaaactttttatttgagcaatatgagcccacaagttggacgaggattcaatcgcgataagtggaatgatttggaaatgcactgcagaagagtcgcaaaaaagatgatcaactcctacataatcaccggtcccctctaccttccaaaacttgaaggagatggaaaaaagtatatcaagtatcaagttatcggtgataataacgttgcagtgccaactcatttcttcaaagttgctcttttcgaagtgacacccggcaaattcgagctggaatcctatattttgccaaacgcagtcatcgaggatactgtagaaatttccaagttccatgttccattggatgctgtggagagaagtgcgggtcttgagattttcgctcgtttggaccctaagagcattgtgaaagagaacggagcaaagaagggaggattactttggtgattgattggattgtgattccggtgaattttaggtttaggttgttttgtttttgtttcacaaataaattttttgttaa

SEQ ID NO: 4 - protein, 308 aa, Caenorhabditis elegans
MIGKVAGTAAIAGISFLAGKYSNDDLPIFRNVQSATNVPMNQIQVSEPMTVKPASLNADAMGPSRSAEIMKHGYPGFTNVRTYEDFVLSYDYKTRTAHWVCEHLTPERLKHAEGVDRKLCEFKPDITFPQKFLSQNTDYKCSGFDRGHLAAAGNHRKSQLAVDQTFYLSNMSPQVGRGFNRDKWNDLEMHCRRVAKKMINSYIITGPLYLPKLEGDGKKYIKYQVIGDNNVAVPTHFFKVALFEVTPGKFELESYILPNAVIEDTVEISKFHVPLDAVERSAGLEIFARLDPKSIVKENGAKKGGLLW

(19) United States
(12) Patent Application Publication
(10) Pub. No.: US 2025/0346550 A1
(43) Pub. Date: Nov. 13, 2025

(54) METHODS OF MODULATING ENDONUCLEASE G USING RESVERATROL AND ITS DERIVATIVES

(71) Applicant: THE REGENTS OF THE UNIVERSITY OF COLORADO A BODY CORPORATE, Denver, CO (US)

(72) Inventors: Ding XUE, Boulder, CO (US); Michael STOWELL, Boulder, CO (US); Hanna Shiao-Han YUAN, TAIPEI (TW); Graham REDWEIK, Boulder, CO (US); Jason Liang-Ju LIN, TAIPEI (TW); Xiaoqi WU, Ann Arbor, MI (US)

(21) Appl. No.: 18/870,343

(22) PCT Filed: Jun. 1, 2023
(86) PCT No.: PCT/US2023/067801

§ 371 Date: Nov. 27, 2024

Related U.S. Application Data
(60) Provisional application No. 63/348,434, filed on Jun. 2, 2022.

(51) Int. Cl. C07C 49/747; A61K 31/122; A61K 31/15; A61P 25/16; A61P 25/28

(52) U.S. Cl. CPC C07C 49/747; A61K 31/122; A61K 31/15; A61P 25/16; A61P 25/28

(57) ABSTRACT

The present invention describes the mechanism and physiological target for resveratrol, and in particular the novel mechanism by which trans- and c/.s-resveratrol isomers show opposite effects in enhancing and inhibiting the nuclease activity of EndoG in vitro and multiple important cellular processes in vivo.

### CROSS-REFERENCE TO RELATED APPLICATIONS

This International PCT application claims the benefit of and priority to U.S. Provisional Application No. 63/348,434 filed Jun. 2, 2022, the specification, claims and drawings of which are incorporated herein by reference in their entirety.

### GOVERNMENT INTEREST

This invention was made with Government support under grant numbers R01 GM118188 and F32NS124826 awarded by the National Institutes of Health (NIH). The U.S. Government has certain rights in this invention.

### SEQUENCE LISTING

The instant application contains contents of the electronic sequence listing (90245.00821-Sequence-Listing.xml; Size: 7,321 bytes; and Date of Creation: Jun. 1, 2023) is herein incorporated by reference in its entirety.

### TECHNICAL FIELD

The invention provides novel compositions and methods for modulating the activity of mitochondrial endonuclease G (EndoG). In particular, the invention describes the action of cis- and trans-resveratrol to inhibit or increase the nuclease activity of EndoG, respectively. In additional embodiments, the invention provided novel compositions and methods of treating cancer in a subject. In particular, the invention describes the action of cis-resveratrol, and preferably a stabilized cis-resveratrol isomer to treat cancer.

### BACKGROUND

Mitochondria are unique organelles comprised of a double membrane and are responsible for generating adenosine triphosphate (ATP), the major energy source in the cell. In addition, they are critical for many cellular processes, including cellular respiration, apoptosis, metabolism, and stress response. Unlike most organelles in the cell, mitochondria are unique in having their own genomes (mtDNA), which are circular DNA and encode 2 ribosomal RNAs, 22 tRNAs, and 13 respiratory chain proteins. In most eukaryotes, mitochondria and their genomes are inherited maternally, with paternal mitochondria selectively eliminated following fertilization. How paternal mitochondria are selectively eliminated during development has been a topic of great interest. Maternal degradation machineries, autophagosomes and proteosomes, have been implicated in paternal mitochondrial elimination (PME).

In addition, paternal mitochondria are depolarized and undergo rapid internal breakdown following fertilization in C. elegans. This internal breakdown of paternal mitochondria is catalyzed by the CPS-6 protein (SEQ ID NO. 3, 4), a C. elegans homologue of the mammalian mitochondrial endonuclease G (EndoG) (SEQ ID NO. 1, 2). CPS-6 normally localizes in the intermembrane space of mitochondria and relocates to the matrix of paternal mitochondria following fertilization and their rapid internal breakdown, which promotes degradation of mtDNA and autophagosome enclosure of sperm mitochondria, leading to degradation and removal of paternal mitochondria. Therefore, paternal and maternal degradation pathways interact and coordinate to promote rapid PME.

Why paternal mitochondria are selectively removed during embryo development is poorly understood. Delayed removal of paternal mitochondria due to loss of CPS-6 has been shown to slow cell divisions and cause increased embryonic lethality, indicating that abnormal persistence of paternal mitochondria interferes with and has an adverse effect on animal development. There are also rare examples of abnormal transmission of paternal mitochondria in humans, and in each case the coexistence of maternal and paternal mitochondria in the so-called heteroplasmy state causes various clinical symptoms, including fatigue, muscle-related deficiencies, and developmental delay. These observations are consistent with findings in C. elegans and underscore the importance of eliminating paternal mitochondria to ensure normal development and cellular functions. Currently there is no treatment for patients carrying persistent paternal mitochondria, which remains an important unmet medical need.

To address this problem, the present inventors set out to screen for small molecules that could modulate PME and have focused on natural compounds derived from fruits and vegetables using a candidate approach. Resveratrol, a compound derived from fruits and plants, was shown dramatically alter PME. Importantly, resveratrol exists as a mixture of trans- and cis-isomers, which can be interconverted by light absorption. Furthermore, the present inventors unexpectedly observe that these two isomers exhibit opposite effects on PME, with trans-isomer enhancing and cis-isomer inhibiting PME, respectively. Moreover, we find that both isomers specifically target CPS-6, a mitochondrial endonuclease G and a crucial PME executor, to affect its endonuclease activity in vitro and PME in vivo. Resveratrol isomers are thus the first identified compounds that can both positively and negatively modulate mitochondrial inheritance and potentially can be used to treat diseases caused by dysregulated mitochondrial inheritance or diseases due to altered EndoG activity.

As noted above, EndoG plays important roles in multiple different biological processes in diverse organisms, including apoptosis, paternal mitochondrial and mtDNA elimination during development, neurodegeneration, autophagy, mtDNA replication, and mitochondrial maintenance. Reduction or loss of EndoG has been shown to delay or block apoptosis, PME, autophagy, and neurodegeneration and can cause cardiac hypertrophy. In addition, the present inventors have demonstrated that the cis-resveratrol isomer, and preferably a stabilized cis-resveratrol isomer can effectively treat cancer cells. Such a result is highly counter-intuitive as the inhibition of EndoG, which promotes apoptosis, would not appear to be an effective anti-cancer agent.

### SUMMARY OF THE INVENTION

The present invention provides, in part, a method for treating a disease or disorder, and preferably EndoG-associated diseases, including neurodegeneration and PME-related diseases, such as subject carrying persistent paternal mitochondria. In a preferred embodiment, the invention includes administering to a subject in need thereof a therapeutically effective amount of a resveratrol isomer or their stabilized derivatives.

The present invention further provides, in part, a method for treating a disease or disorder, and preferably cancer, comprising administering to a subject in need thereof a therapeutically effective amount of a substantial isolated or substantially pure quantity of a cis-resveratrol isomer, or a stabilized cis-resveratrol isomer.

The present invention further provides, in part, a method for treating a disease or disorder, and preferably cancer, comprising administering to a subject in need thereof a therapeutically effective amount of a cis-resveratrol isomer, or a stabilized cis-resveratrol isomer.

In one preferred embodiment, the invention includes a novel resveratrol derivative stabilized in the cis-configuration by a 5-member aromatic ring having a ketone group (2-Cyclopentanone). In this embodiment, the stabilized cis-resveratrol isomer compound according to Formula IV:

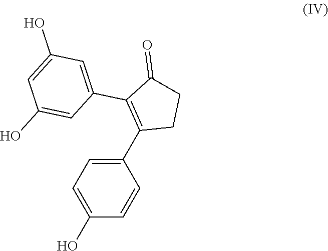 [SMILES: O=C1CCC(c2ccc(O)cc2)=C1c1cc(O)cc(O)c1]

In one preferred embodiment, the invention includes methods of synthesizing stabilized cis-resveratrol isomer compound according to Formula III.

In one preferred embodiment, the invention includes an aza-resveratrol derivative stabilized into a trans-configuration. In this embodiment, the stabilized trans-resveratrol isomer comprises a compound according to Formula IV:

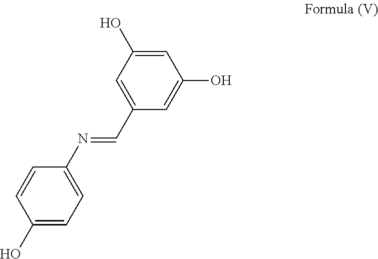 [SMILES: Oc1ccc(/N=C/c2cc(O)cc(O)c2)cc1]

The present invention further provides, in part, novel methods and compositions for modulating the activity of endonuclease G (EndoG) comprising a resveratrol isomer. In one preferred embodiment, a resveratrol isomer, and preferably a cis-resveratrol, or stabilized cis-resveratrol isomer inhibits the activity of EndoG in vitro, or in vivo. In another preferred embodiment, a resveratrol isomer, and preferably a trans-resveratrol, or stabilized trans-resveratrol isomer increases the activity of EndoG in vitro, or in vivo.

The present invention further provides, in part, pharmaceutical compositions comprising one or more resveratrol isomers, and a pharmaceutically acceptable carrier. In one preferred embodiment, a pharmaceutical composition of the invention includes a therapeutically effective amount of a substantially isolated or substantially pure a cis- or trans-resveratrol isomer, and a pharmaceutically acceptable carrier. In another preferred embodiment, a pharmaceutical composition of the invention includes a therapeutically effective amount of a stabilized cis- or trans-resveratrol isomer, and a pharmaceutically acceptable carrier.

The present invention further provides, in part, a pharmaceutical composition of the invention is provided in a kit, including a container for a composition and instructions for administration of the composition.

The present invention further provides, in part, a method for treating a disease or disorder, and preferably an EndoG related disease or condition, comprising administering to a subject in need thereof a therapeutically effective amount of a substantial isolated or substantially pure quantity of a cis-resveratrol isomer, or a stabilized cis-resveratrol isomer.

The present invention further provides, in part, a method for treating a disease or disorder, and preferably an EndoG-related disease or condition, such as neurodegeneration, and in particular α-synuclein-induced neurodegeneration, comprising administering to a subject in need thereof a therapeutically effective amount of a substantial isolated or substantially pure quantity of a cis-resveratrol isomer, a stabilized cis-resveratrol isomer or a stabilized trans-resveratrol isomer.

The present invention further provides, in part, a method for treating a disease or disorder, and preferably an PME related disease or condition, comprising administering to a subject in need thereof a therapeutically effective amount of a substantial isolated or substantially pure quantity of a trans-resveratrol isomer, or a stabilized trans-resveratrol isomer.

The present invention further provides, in part, a method for treating a disease or disorder, and preferably an PME related disease or condition, such as the persistent paternal mitochondria or damaged mitochondria, cardiac hypertrophy, and diseases caused by autophagy deficiency comprising administering to a subject in need thereof a therapeutically effective amount of a substantially isolated or substantially pure quantity of a trans-resveratrol isomer, or a stabilized trans-resveratrol isomer.

Additional aspects of the invention may become evident based on the specification and figures presented below.

### BRIEF DESCRIPTION OF THE DRAWINGS

FIG. 1. Interconversion of two resveratrol isomers and their opposing effects on PME. a, A screen for compounds that affect PME. The numbers of MTR-stained paternal mitochondrial clusters in 64-cell cross-fertilized embryos from mating of MTR-stained N2 males with N2 hermaphrodites in the presence of the indicated compounds (100 μM) were scored. n≥15 embryos for each compound. b, d-g, Quantification of MTR-stained paternal mitochondrial clusters in 64-cell stage embryos (b, d-f) or in embryos at the indicated stage (g) from the cross between MTR-stained N2 males and N2 hermaphrodites, in the presence of 0.25% ethanol (Mock) or 50 μM of the indicated compound. In each experiment, n>40 embryos (b,d,e), n=20 embryos (f), and n≥10 embryos (g) were scored, respectively. In b, fresh resveratrol indicates that resveratrol was prepared freshly. Aged resveratrol indicates that resveratrol was reused multiple times and had been exposed to light. In e and f, the indicated compound was irradiated with ultraviolet (UV) for 4 hours before being used to treat animals. c, Interconversion of trans-resveratrol and cis-resveratrol after exposure to UV or light. Data are mean±s.e.m. **P<0.01, ***P<0.001; n.s., not significant, two-sided, unpaired t-test.

FIG. 2. High performance liquid chromatography analysis of resveratrol isomers. a, b, e-g HPLC profiles and peak retention times detected at 303 nm of 1.0 μg commercially “pure” trans-resveratrol (a), 1.0 μg of commercially “pure” cis-resveratrol (b), 20 μL of 50 μM aged trans-resveratrol solution (e), 20 μL of 50 μM commercially “pure” trans-resveratrol solution subjected to UV irradiation for 4 hours at 0° C. (f), and 20 μL of 50 μM commercially “pure” cis-resveratrol solution subjected to UV irradiation for 4 hours at 0° C. (g). Due to the different extinction coefficients of the resveratrol isomers, standard curves of trans-resveratrol (c) and cis-resveratrol (d) were generated by analyzing a series of working solutions for each isomer. Good linear correlations (R²≥0.9999) were acquired over the concentration range of 0-45 μM trans-resveratrol (c) and 0-110 μM cis-resveratrol (d), with the corresponding regression equations. (h) Compositions of the resveratrol isomers in the indicated samples, without or with UV irradiation (see Methods) as quantified by HPLC.

FIG. 3. Resveratrol isomers target CPS-6 to affect PME. a, b, Both cis-resveratrol and trans-resveratrol act paternally to affect PME. The numbers of MTR-stained paternal mitochondrial clusters in embryos at 64-cell (a) or 16-cell (b) stage from the indicated crosses were scored. N2 males or hermaphrodites were pretreated with 0.25% ethanol (Mock), 50 μM cis-resveratrol (a), or 50 μM trans-resveratrol (b) as indicated before mating. c-g, Quantification of MTR-stained paternal mitochondrial clusters in 64-cell embryos from the indicated crosses treated with 0.25% ethanol (Mock), 50 μM cis-resveratrol, and 50 μM trans-resveratrol, respectively. Alleles used are lgg-1(bp523), cps-6(tm3222) and rad-23(tm2595). Data are mean s.e.m; n≥15 embryos (a) and n≥20 embryos (b, c-g) were scored in each experiment. ***P<0.001; n.s., not significant, two-sided, unpaired t-test. h, Resveratrol isomers enhance or inhibit the nuclease activity of CPS-6 in a plasmid DNA cleavage assay. 10 ng of CPS-6 were incubated with 0.25% ethanol (Mock), 20 μM cis-resveratrol, or 20 μM trans-resveratrol in dark at 4° C. for 1.5 hours, before 1 μg of the pPD49.78 plasmid DNA were added to the reactions. After further incubation at 30° C. for 1 hour, the reactions were resolved on a 1% agarose gel. Three different forms of plasmid DNA are indicated.

FIG. 4. Resveratrol isomers affect autophagosome formation on paternal mitochondria. a-f, Analysis of enclosure of MTR-stained paternal mitochondria by LGG-1 autophagosomes. Zygotes from the indicated crosses with MTR-stained N2 males pretreated with 0.25% ethanol (Mock)(a,b), 50 μM trans-resveratrol (c,d), or 50 μM cis-resveratrol (e,f) were labeled with an antibody to LGG-1. Images were acquired with a Nikon SIM microscope. Dashed rectangles highlight the areas enlarged and shown below (b,d,e). Scale bars, 2 μm (a,c,e), 0.5 μm (b,d,f). g, Quantification of four types of paternal mitochondria in zygotes that were fully enclosed, partially enclosed, and not enclosed (isolated) by the LGG-1 autophagosomes and that had initiating phagophore, respectively. The number of MTR-stained paternal mitochondria scored in each experiment was shown below the pie chart.

FIG. 5. Resveratrol isomers target CPS-6 to affect α-synuclein-induced dopaminergic neuronal death. a, Loss of cps-6 blocked α-synuclein-induced dopaminergic neuron loss in adult day 2 baIn11 animals. b, 100 μM cis-resveratrol (cis) strongly inhibited and 100 μM trans-resveratrol (trans) enhanced dopaminergic neuronal death in adult day 2 baIn11 animals, respectively, compared to 0.25% ethanol treatment (Mock), but did not cause any dopaminergic neuronal loss in cps-6(tm3222); baIn11 animals. In each experiment, 300 animals were scored to determine the percentage of animals losing at least one dopaminergic neuron in the head. Data are mean±s.e.m. *P<0.05; ***P<0.001, two-sided, unpaired t-test.

FIG. 6. Analysis of the effects of antioxidant compounds on PME. a, The chemical structures of 12 different antioxidant compounds screened in this study. b, c, Analysis of the effects of six compounds on PME in the indicated crosses. The numbers of MTR-stained paternal mitochondrial clusters were scored as in FIG. 1a. cps-6(tm3222) and lgg-1(bp523) alleles were used in the assays. Data are mean±s.e.m. n=20 embryos (b) and 15 embryos (c) in each experiment. **P<0.01; ***P<0.001; n.s., not significant, two-sided, unpaired t-test.

FIG. 7. Effects of different dosages of resveratrol isomers on PME. a, b, The effects of different dosages of cis-resveratrol (a) and trans-resveratrol (b) on PME. The numbers of MTR-stained paternal mitochondrial clusters in 64-cell embryos (a) or 16-cell embryos (b) from mating of MTR-stained N2 males with N2 hermaphrodites in the presence of the indicated concentrations of resveratrol isomers were scored. Data are mean±s.e.m. in =20 embryos in each experiment. ***P<0.001; n.s., not significant, two-sided, unpaired t-test. c, A diagram of C. elegans mtDNA, the uaDf5 deletion, primers used in the nested PCR assays, and sizes of PCR products in N2 and uaDf5/+ animals. d-f, Resveratrol isomers affect elimination of uaDf5 paternal mtDNA. N2 hermaphrodites and MTR-stained uaDf5/+ males were mated as indicated in the presence of 0.25% ethanol (Mock, d), 50 μM trans-resveratrol (e), or 50 μM cis-resveratrol (f). Males also carried smIs42, an integrated Pₛᵤᵣ₋₅sur-5::gfp transgene, which directs GFP expression in all somatic cells in most developmental stages, and was used to track cross progeny. A single cross-fertilized embryo or larva (MTR- or GFP-positive) at the indicated stage was analyzed by PCR. uaDf5/+ and N2 hermaphrodites were controls.

FIG. 8. Resveratrol isomers target CPS-6 to affect α-synuclein-induced dopaminergic neuronal death. a, Loss of cps-6 blocked α-synuclein-induced dopaminergic neuron loss in adult day 2 baIn11 animals. b, 100 μM cis-resveratrol (cis) strongly inhibited and 100 μM trans-resveratrol (trans) enhanced dopaminergic neuronal death in adult day 2 baIn11 animals, respectively, compared to 0.25% ethanol treatment (Mock), but did not cause any dopaminergic neuronal loss in cps-6(tm3222); baIn11 animals. c, 50 μM of stabilized trans-resveratrol also enhanced DA neuronal death in adult day 2 baIn11 animals. In each experiment, 300 animals were scored to determine the percentage of animals losing at least one dopaminergic neuron in the head. Data are mean±s.e.m. *P<0.05; ***P<0.001, two-sided, unpaired t-test.

FIG. 9. Cis-resveratrol inhibits and trans-resveratrol promotes germline tumor formation. Tumor size and tumor occurring frequency of the daf-16(mu86); cki-2(0k2105); glp-1 (ar202) mutant grown at 20° C. and treated with 1% ethanol (Mock), 200 μM cis-resveratrol (Cis), and 200 μM trans-resveratrol (Trans), respectively, are shown. Tumor assays were performed in triplicate with 100 animals per replicate. a, A five-stage classification was created to measure the tumor size in adult day 5 animals under the Normaski optic. Score 1 denotes the normal gonads seen in wild-type animals. Score 5 denotes enlarged gonads full of mitotic germ cells and without any egg or oocyte. Score 2-4 denote partially enlarged gonads. b, Tumor frequency indicates the percentage of animals with enlarged gonads (score 5). Data from three independent biological replicates are shown as mean±SEM. *P<0.05; **P<0.01; ***P<0.001, two-sided, unpaired t-test.

FIG. 10. Trans-resveratrol reduces embryonic lethality through enhancing removal of mutant paternal mitochondria. The embryonic lethality rate was scored in cross-fertilized embryos from crosses of the indicated genotypes. Males were pretreated with either 0.5% ethanol (Mock) or 100 WM trans-resveratrol (trans) for eight hours before mating. All males carried smIs42 to assist identification of zygotes. Data are means SEM; n>200 embryos per cross at 25° C. n.s., no significant difference; *P<0.05 using two-sided, unpaired t-test.

FIG. 11. Stabilized trans-resveratrol enhances the nuclease activity of CPS-6 in a plasmid DNA cleavage assay and is resistant to UV irradiation. 10 ng of CPS-6 were incubated with 0.25% ethanol (Mock), 20 μM locked trans-resveratrol with or without UV irradiation, 20 μM trans-resveratrol with or without UV irradiation, and 20 μM cis-resveratrol with or without UV irradiation, respectively, in dark at 4° C. for 1.5 hours, before 1 μg of the pPD49.78 plasmid DNA were added to the reactions. After further incubation at 30° C. for 1 hour, the reactions were resolved on a 1% agarose gel. Three different forms of plasmid DNA are indicated. Resveratrol isomers or derivative were subjected to UV irradiation for 3 hours.

FIG. 12. Stabilized trans-resveratrol enhances PME as well as trans-resveratrol. Quantification of MTR-stained paternal mitochondrial clusters at the indicated embryonic stages from the cross between MTR-stained N2 males and N2 hermaphrodites, in the presence of 0.25% ethanol (Mock) or 50 μM of the indicated compound. In each experiment, n≥10 embryos embryos. Data are mean±s.e.m. **P<0.01, ***P<0.001; n.s., not significant, two-sided, unpaired t-test.

FIG. 13. Stabilized cis-resveratrol inhibits α-synuclein-induced dopaminergic neuronal death as well as cis-resveratrol. cis-resveratrol and Stabilized cis-resveratrol (100 μM) showed comparable activity in strongly inhibiting dopaminergic neuronal death in adult day 2 baIn11 animals, respectively, compared to 0.25% ethanol treatment (Mock). In each experiment, 300 animals were scored to determine the percentage of animals losing at least one of the six dopaminergic neurons in the head. Data are mean±s.e.m. ****P<0.001, two-sided, unpaired t-test.

FIG. 14. stabilized cis-resveratrol inhibits germline tumor formation as well as cis-resveratrol. Tumor occurring frequency of the daf-16 (mu86); cki-2 (0k2105); glp-1 (ar202) mutant (ET507) grown at 20° C. and treated with 0.25% ethanol (Mock), 100 μM cis-resveratrol, and 100 μM Stabilized cis-resveratrol, respectively, are shown. Tumor assays were performed in triplicate with 100 animals per replicate. Data are mean±s.e.m. ****P<0.001, two-sided, unpaired t-test

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

In eukaryotes, mitochondria and their genomic DNA (mtDNA) are inherited maternally in most species. Abnormal persistence of paternal mitochondria is detrimental to normal development and causes various deficiencies, including neurological and muscular defects, which lack treatment options. The present inventors performed a candidate-based screen to seek compounds affecting paternal mitochondrial elimination (PME) in Caenorhabditis elegans (C. elegans) and identified a plant-derived natural compound, resveratrol. Resveratrol normally exists as a mixture of trans- and cis-isomers, which are readily interconverted by light absorption. Interestingly, trans and cis resveratrol isomers show opposite effects in promoting and inhibiting PME, respectively. Chemical genetic analysis reveals that resveratrol isomers target the paternal PME pathway mediated by CPS-6, a C. elegans mitochondrial endonuclease G (EndoG). Biochemical analysis demonstrates that trans-resveratrol enhances and cis-resveratrol inhibits the endonuclease activity of CPS-6. Cell biological analysis indicates that resveratrol isomers affect autophagosome formation on paternal mitochondria. Moreover, trans-resveratrol and cis-resveratrol target CPS-6 to enhance and inhibit dopaminergic neuronal death in a C. elegans Parkinson's disease model caused by overexpression of human alpha-synuclein, respectively. the present invention demonstrates unexpected, opposing activities of resveratrol isomers in regulating the nuclease activity of EndoG and the associated PME and neurodegeneration processes, which could be the cause of toxicity and adverse side effects seen in resveratrol treatments. In one embodiment of the present invention, resveratrol isomers or their stabilized derivatives, can be used to inhibit EndoG in a subject in need thereof, and further treat EndoG-associated diseases, including PME-related diseases and neurodegeneration.

The present invention provides, in part, a method for treating a disease or disorder, and preferably EndoG-associated diseases, including neurodegeneration and PME-related diseases, such as subject carrying persistent paternal mitochondria. In a preferred embodiment, the invention includes administering to a subject in need thereof a therapeutically effective amount of a resveratrol isomer or their stabilized derivatives.

As used herein, “resveratrol” means 3,5,4′-trihydroxy-trans-stilbene. Resveratrol isomer means cis-resveratrol isomer or trans-resveratrol isomer, or a stabilized cis-resveratrol isomer or a stabilized trans-resveratrol isomer, are generally and collectively sometimes referred to as a compound of the invention or composition of the invention.

As used herein, a cis-resveratrol isomer means the compound, and preferably a substantially isolated or pure compound, having the following chemical structure:

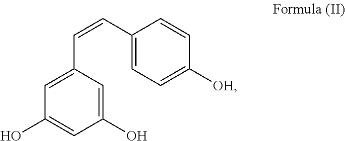 [SMILES: Oc1ccc(/C=C\c2cc(O)cc(O)c2)cc1]

or a pharmaceutically acceptable salt thereof.

As used herein, a trans-resveratrol isomer means the compound, and preferably a substantially isolated or pure compound, having the following chemical structure:

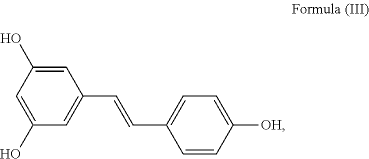 [SMILES: Oc1ccc(/C=C/c2cc(O)cc(O)c2)cc1]

or a pharmaceutically acceptable salt thereof.

As used herein, a stabilized cis-resveratrol isomer means the compound having the following chemical structure:

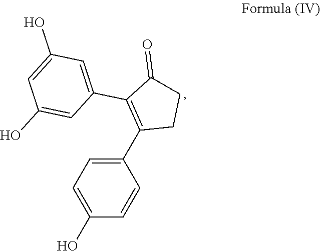 [SMILES: O=C1CCC(c2ccc(O)cc2)=C1c1cc(O)cc(O)c1]

or a pharmaceutically acceptable salt thereof.

In certain embodiments, the invention includes methods of synthesizing stabilized cis-resveratrol isomer compound according to formula (IV).

In one preferred embodiment, the invention includes an resveratrol derivative stabilized into a trans-configuration through an aza substitution. In this embodiment, the aza-resveratrol derivative, generally referred to herein as a stabilized trans-resveratrol isomer; comprises a compound according to Formula IV:

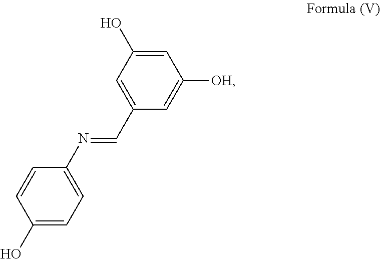 [SMILES: Oc1ccc(/N=C/c2cc(O)cc(O)c2)cc1]

or a pharmaceutically acceptable salt thereof.

In certain embodiments, the invention includes methods of synthesizing stabilized trans-resveratrol isomer compound according to formula (V).

Additional embodiments of the current invention include a compound of Formula I-V, or a pharmaceutically acceptable salt, solvate, stereoisomer, tautomer, or prodrug thereof, for use in recreational, psychological, for use in a medical therapy.

Additional embodiments of the current invention include a substantially isolated or substantially pure compound of Formula II-III, or a pharmaceutically acceptable salt, solvate, stereoisomer, tautomer, or prodrug thereof, for use in a medical therapy.

One embodiment of the present invention provides a systems, methods, and compositions for novel resveratrol isomers according to the compounds of Formula IV-V, and a pharmaceutically acceptable carrier or diluent, which may preferably further include a method of treatment of the human or animal body using one or more of the novel compounds, or pharmaceutical compositions described herein.

One embodiment of the present invention provides a systems, methods, and compositions for a substantially isolated or substantially pure resveratrol isomers, a preferably a cis- or trans-resveratrol isomer according to the compounds of Formula II-III, and a pharmaceutically acceptable carrier or diluent, which may preferably further include a method of treatment of the human or animal body using one or more of the compounds, or pharmaceutical compositions described herein.

One embodiment of the present invention provides a method for treating a disease or condition for which modulation of EndoG activity is beneficial comprising the steps of administering to a subject in need thereof, a therapeutically effective amount of a novel resveratrol isomers according to the compounds of Formula IV-V, or a pharmaceutically acceptable salt thereof.

One embodiment of the present invention provides a method for treating a disease or condition for which inhibition of EndoG activity is beneficial comprising the steps of administering to a subject in need thereof, a therapeutically effective amount of a novel cis-resveratrol isomer according to the compounds of Formula IV, or a pharmaceutically acceptable salt thereof.

One embodiment of the present invention provides a method for treating cancer is beneficial comprising the steps of administering to a subject in need thereof, a therapeutically effective amount of a novel cis-resveratrol isomer according to the compounds of Formula IV, or a pharmaceutically acceptable salt thereof. A method for treating cancer comprising the steps of administering to a subject in need thereof, a therapeutically effective amount of a combination comprising a novel cis-resveratrol isomer according to the compounds of Formula IV, and an anti-cancer therapeutic composition.

One embodiment of the present invention provides a method for treating a disease or condition for which increasing EndoG activity is beneficial comprising the steps of administering to a subject in need thereof, a therapeutically effective amount of a novel trans-resveratrol isomer according to the compounds of Formula V, or a pharmaceutically acceptable salt thereof.

One embodiment of the present invention provides a method for treating a disease or condition for which modulation of EndoG activity is beneficial comprising the steps of administering to a subject in need thereof, a therapeutically effective amount of a substantially isolated or substantially pure resveratrol isomer according to the compounds of Formula II-III, or a pharmaceutically acceptable salt thereof.

One embodiment of the present invention provides a method for treating a disease or condition for which inhibition of EndoG activity is beneficial comprising the steps of administering to a subject in need thereof, a therapeutically effective amount of a substantially isolated or substantially pure cis-resveratrol isomer according to the compounds of Formula II, or a pharmaceutically acceptable salt thereof.

One embodiment of the present invention provides a method for treating cancer is beneficial comprising the steps of administering to a subject in need thereof, a therapeutically effective amount of a substantially isolated or substantially pure cis-resveratrol isomer according to the compounds of Formula II, or a pharmaceutically acceptable salt thereof. A method for treating cancer comprising the steps of administering to a subject in need thereof, a therapeutically effective amount of a combination comprising a substantially isolated or substantially pure cis-resveratrol isomer according to the compounds of Formula II, and an anti-cancer therapeutic composition.

One embodiment of the present invention provides a method for treating a disease or condition for which increasing EndoG activity is beneficial comprising the steps of administering to a subject in need thereof, a therapeutically effective amount of a substantially isolated or substantially pure trans-resveratrol isomer according to the compounds of Formula III, or a pharmaceutically acceptable salt thereof.

The present invention further provides, in part, a method for treating a disease or disorder, and preferably an EndoG related disease or condition, comprising administering to a subject in need thereof a therapeutically effective amount of a substantial isolated or substantially pure quantity of a cis-resveratrol isomer, or a stabilized cis-resveratrol isomer.

The present invention further provides, in part, a method for treating a disease or disorder, and preferably an EndoG-related disease or condition, such as neurodegeneration, and in particular α-synuclein-induced neurodegeneration, comprising administering to a subject in need thereof a therapeutically effective amount of a substantial isolated or substantially pure quantity of a cis-resveratrol isomer, or a stabilized cis-resveratrol isomer.

The present invention further provides, in part, a method for treating a disease or disorder, and preferably an PME related disease or condition, comprising administering to a subject in need thereof a therapeutically effective amount of a substantial isolated or substantially pure quantity of a trans-resveratrol isomer, or a stabilized trans-resveratrol isomer.

The present invention further provides, in part, a method for treating a disease or disorder, and preferably an PME related disease or condition, such as such as the persistent paternal mitochondria or damaged mitochondria, cardiac hypertrophy, diseases caused by autophagy deficiency comprising administering to a subject in need thereof a therapeutically effective amount of a substantial isolated or substantially pure quantity of a trans-resveratrol isomer, or a stabilized trans-resveratrol isomer.

Unless indicated otherwise, all references herein to a resveratrol isomer, and in particular cis- and trans-resveratrol isomers, and stabilized cis- and trans-resveratrol isomers include references to pharmaceutically acceptable salts, solvates, hydrates and complexes thereof, and to solvates, hydrates and complexes of pharmaceutically acceptable salts thereof, and include amorphous and polymorphic forms, stereoisomers, and isotopically labeled versions thereof.

It may be convenient or desirable to prepare, purify, and/or handle a corresponding solvate of the active compound. The term “solvate” is used herein in the conventional sense to refer to a complex of solute (e.g., active compound, salt of active compound) and solvent. If the solvent is water, the solvate may be conveniently referred to as a hydrate, for example, a mono-hydrate, a di-hydrate, a tri-hydrate, etc. It may be convenient or desirable to prepare, purify, and/or handle the active compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999). For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

One or more of the compounds of the invention may exist in the form of pharmaceutically acceptable salts such as, e.g., acid addition salts and base addition salts of the compounds of one of the compound(s) identified herein. As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the compounds of the invention identified herein.

The invention also relates to prodrugs of the compounds of the formulae provided herein. Thus, certain derivatives of compounds of the invention which may have little or no pharmacological activity themselves can, when administered to a patient, be converted into the inventive compounds, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association), the disclosures of which are incorporated herein by reference in their entireties.

Prodrugs in accordance with the invention can, for example, be produced by replacing appropriate functionalities present in the inventive compounds with certain moieties known to those skilled in the art as ‘pro-moieties’ as described, for example, in “Design of Prodrugs” by H Bundgaard (Elsevier, 1985), the disclosure of which is incorporated herein by reference in its entirety.

The term “modulation,” or “modulate” as used herein in the context of the activity of an enzyme, and preferably an EndoG enzyme, refers to a change in activation state as compared to the absence of a compound of the invention.

As used herein, “inhibits,” “inhibition” refers to the decrease in activity of a target protein product relative to the normal wild-type level. Inhibition may result in a decrease in activity of a target enzyme, and preferably a EndoG, and more preferably a decrease in the nuclease activity of EndoG in response to a cis-resveratrol isomer or a stabilized cis-resveratrol isomer of the invention by less than 10%, 10%, 15%, 20%, 25%, 30%, 35%, 40%, 45%, 50%, 55%, 60%, 65%, 70%, 75%, 80%, 85%, 90%, 95% or 100%.

As used herein, “increase,” “increasing” refers to the increase in activity of a target protein product relative to the normal wild-type level. Increasing may result in an increase in activity of a target enzyme, and preferably a EndoG, and more preferably an increase in the nuclease activity of EndoG in response to a trans-resveratrol isomer or a stabilized trans-resveratrol isomer of the invention by less than 10%, 10%, 15%, 20%, 25%, 30%, 35%, 40%, 45%, 50%, 55%, 60%, 65%, 70%, 75%, 80%, 85%, 90%, 95% or 100%.

As used herein, “substantially isolated” means that the in a heterogenous mixture containing both cis-resveratrol and trans-resveratrol isomers of the invention, one of the isomers has been substantially separated or converted into the other isoform, such that one of the resveratrol isoforms comprises a majority of the isomer species of the mixture. For example, in one embodiment, a pharmaceutical composition containing a “substantially isolated” amount of a cis-resveratrol isomer, includes a quantity wherein the cis-resveratrol isomer comprises a majority of the isomer species present, which may include greater than 50%, about 60%, about 70%, about 80%, about 90%, about 95%, about 98%, or about 99%.

As used herein, “substantially pure” means that a cis-resveratrol or trans-resveratrol isomer of the invention is provided in a form that is homogenous for one species. For example, in one embodiment, a pharmaceutical composition containing a substantially pure amount of a cis-resveratrol isomer, includes a quantity wherein the cis-resveratrol isomer is the only detectable isomer.

As used herein “endonuclease G” or “EndoG” refers to a nuclear-encoded mitochondrial nuclease that has been reported to function in apoptosis, DNA recombination and cell proliferation. The protein encoded by this gene is a nuclear encoded endonuclease that is localized in the mitochondrion. The encoded protein is widely distributed among animals and cleaves DNA at GC tracts. This protein is capable of generating the RNA primers required by DNA polymerase gamma to initiate replication of mitochondrial DNA. (Côté, J. and Ruiz-Carrillo, A. (1993) Science 261, 765-769; Parrish, J. et al. (2001) Nature 412, 90-94.; Li, L. Y. et al. (2001) Nature 412, 95-99; Zhang, J. et al. (2003) Proc. Natl. Acad. Sci. USA 100, 15782-15787 and Huang, K. J. et al. (2006) Proc. Natl. Acad. Sci. USA 103, 8995-9000. Homo sapiens endonuclease G, mRNA (cDNA clone MGC:4842 complete cds and its sequence has for instance been described by Strausberg, R. L. et al. in Proc. Natl. Acad. Sci. U.S.A. 99 (26), 16899-16903 (2002) and the Homo sapiens endonuclease G (ENDOG), nuclear gene encoding mitochondrial protein, mRNA and its sequence has for instance been described by Varecha, M. et al. in Apoptosis 12 (7), 1155-1171 (2007).

As used herein, “subject” refers to a human or animal subject. In certain preferred embodiments, the subject is a human.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

As noted above, EndoG plays important roles in multiple different biological processes in diverse organisms, including apoptosis, paternal mitochondrial and mtDNA elimination during development, neurodegeneration, autophagy, mtDNA replication, and mitochondrial maintenance. Reduction or loss of EndoG has been shown to delay or block apoptosis, PME, autophagy, and neurodegeneration and can cause cardiac hypertrophy. Therefore, the following diseases or conditions can be treated by one of the resveratrol (RSV) isomers.

| | | Potential |
|---|---|---|
| Disease | Isomers | Mechanisms |
| Neurodegeneration | cis-RSV or stabilized cis-RSV | Block EndoG |
| (α-synuclein-induced) | | activity |
| Cancer | cis-RSV or stabilized cis-RSV | |
| PME-related diseases | trans-RSV or stabilized trans- | Enhance EndoG |
| | RSV | activity |
| Diseases caused by | trans-RSV or stabilized trans- | Enhance EndoG |
| autophagy defects | RSV | activity |
| cardiac hypertrophy | trans-RSV or stabilized trans- | Enhance EndoG |
| caused by reduced | RSV | activity |
| EndoG activity | | |
| Disease caused by | trans-RSV or stabilized trans- | Enhance EndoG |
| damaged mitochondria | RSV | activity |

As used herein “cancer” refers to any malignant and/or invasive growth or tumor caused by abnormal cell growth. Cancer includes solid tumors named for the type of cells that form them, cancer of blood, bone marrow, or the lymphatic system. Examples of solid tumors include sarcomas and carcinomas. Cancers of the blood include, but are not limited to, leukemia, lymphoma and myeloma. Cancer also includes primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one.

Specific types of cancers that may be treated by the compounds of the invention can include: myeloid leukemia acute or chronic, lymphoblastic leukemia acute or chronic, multiple myeloma, Hodgkin's disease, non-Hodgkin's lymphoma or malignant lymphoma; stomach carcinoma, esophagus carcinoma or adenocarcinoma, pancreas ductal adenocarcinoma, insulinoma, glucagonoma, gastrinoma, small bowel adenocarcinoma, colorectal carcinomas; hepatocellular carcinoma, hepatocellular adenoma; carcinoids, genitourinary tract such as kidney adenocarcinoma, Wilm's tumor, bladder and urethra carcinoma and prostate adenocarcinoma, testis cancer like seminoma, teratoma, teratocarcinoma. Interstitial cell carcinoma; uterus endometrial carcinoma, cervical carcinoma, ovarian carcinoma, vulva and vagina carcinoma, Sertoli-L-eydig cell tumors, melanoma, and fallopian tubes carcinoma; lung, alveolar and bronchiolar carcinomas; brain tumors; skin malignant melanoma, basal cell carcinoma, squamous cell carcinoma and Karposl's sarcoma. Also fibrosarcoma, angiosarcoma and rhabdomyosarcoma of the heart and other malignancies that are familiar to those skilled in the art.

In certain embodiments, trans- or stabilized trans-resveratrol isomers can treat hypertension and/or hypertrophy, and preferably cardiac hypertrophy, in a subject. The terms “hypertension” and “hypertrophy” are well known to those skilled in the art. Hypertension is defined as abnormally high blood pressure; and hypertrophy is defined as the increase in the volume of an organ or tissue due to the enlargement of its component cells. In accordance with the present invention, the hypertension associated, and hypertrophy associated diseases are preferably hypertension-, and hypertrophy-induced diseases, respectively, i.e., the underlying cause of these diseases is hypertension, or hypertrophy. The hypertension associated and hypertrophy associated diseases do not include diseases caused by ischemia. Hypertension associated diseases include, but are not limited to, hypertensive heart disease, hypertensive kidney disease and hypertensive vascular dysfunction. Persistent hypertension leads to the thickening of walls of blood vessel and is one of the major contributory factors for congestive heart failure, cardiac arrhythmia (abnormal heart beat), hypertensive nephropathy (damage to kidney due to chronic high blood pressure) and dysfunction of skeletal muscle. Hypertrophy associated diseases include, but are not limited to, hypertrophic cardiomyopathy, left ventricular hypertrophy, valvular disease, such as aortic stenosis, and skeletal muscle hypertrophy.

As used herein, an “EndoG-related disease or condition” or “EndoG-associated diseases” means describes a disease or condition, preferably in a human, that can be treated or ameliorated by inhibiting the activity of EndoG. In a preferred embodiment, cis-resveratrol, and stabilized cis-resveratrol isomers can inhibit the activity of EndoG and treat an EndoG-related disease or condition, such as neurodegeneration, and in particular α-synuclein-induced neurodegeneration or cancer.

As used herein, a “PME-related disease or condition” describes a disease or condition, preferably in a human, that can be treated or ameliorated by increasing the activity of EndoG. In a preferred embodiment, trans-resveratrol, and stabilized trans-resveratrol isomers can increase the activity of EndoG and treat a PME-related disease or condition, such as the persistent paternal mitochondria or damaged mitochondria through mitophagy to facilitate cellular and organismal health.

Administration of a compound of the invention, may be administered by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens of one or more of the compounds of the invention may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound, for example a compound of the invention, calculated to produce the desired therapeutic effect in association with the required pharmaceutical carrier. The specification for the dosage unit forms are dictated by and directly dependent on (a) the unique characteristics of the compound of the invention and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein.

The amount of a compound of the invention administered will be dependent on the subject being treated, the severity of the disorder or condition, the rate of administration, the disposition of the compound and the discretion of the prescribing physician. However, an effective dosage is typically in the range of about 0.001 to about 100 mg per kg body weight per day, preferably about 0.01 to about 35 mg/kg/day, in single or divided doses. For a 70 kg human, this would amount to about 0.07 to about 7000 mg/day, preferably about 0.7 to about 2500 mg/day. In some instances, dosage levels below the lower limit of the aforesaid range may be more than adequate, while in other cases still larger doses may be used without causing any harmful side effect, with such larger doses typically divided into several smaller doses for administration throughout the day. In one preferred embodiment, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.1 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day. In some cases, the aforesaid dosage examples may describe a dosage range for a combination of compounds of the invention. In alternative embodiments, the aforesaid dosage examples may describe dosage ranges for a compound of the invention individually.

In one preferred embodiment, a therapeutically effective amount or dosage of a compound of the invention may be a dosage of a cis-resveratrol or stabilized cis-resveratrol isomer sufficient to inhibit the nuclease activity of EndoG, or treat cancer in a patient. In another preferred embodiment, a therapeutically effective amount or dosage of a compound of the invention may be a dosage of a trans-resveratrol or stabilized trans-resveratrol isomer sufficient to increase the nuclease activity of EndoG.

As used herein, a “pharmaceutically acceptable carrier” refers to a carrier or diluent that does not cause significant irritation to an organism and does not abrogate the biological activity and properties of the administered compound of the invention. The pharmaceutical acceptable carrier may comprise any conventional pharmaceutical carrier or excipient. The choice of carrier and/or excipient will to a large extent depend on factors such as the mode of administration, the effect of the carrier or excipient on solubility and stability, and the nature of the dosage form.

Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension or emulsion, for topical administration as an ointment or cream or for rectal administration as a suppository. The pharmaceutical composition may be in unit dosage forms suitable for single administration of precise dosages.

Exemplary parenteral administration forms include solutions or suspensions of active compounds in sterile aqueous solutions, for example, aqueous propylene glycol or dextrose solutions. Such dosage forms may be suitably buffered, if desired.

Pharmaceutical compositions suitable for the delivery of compounds of the invention, i.e., the compounds of the invention as described herein, and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The one or more compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, depending on dose, the drug may make up from 1 wt % to 80 wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrants will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet. Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet. Other conventional ingredients include antioxidants, colorants, flavoring agents, preservatives and taste-masking agents. Exemplary tablets contain up to about 80 wt % drug, from about 10 wt % to about 90 wt % binder, from about O wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated. The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety. Solid formulations for oral administration may be formulated to be immediate and/or modified release. Modified release formulations include delayed-, sustained-, pulsed-, controlled, targeted and programmed release. Suitable modified release formulations are described in U.S. Pat. No. 6,106,864. Details of other suitable release technologies such as high energy dispersions and osmotic and coated particles can be found in Verma et al, Pharmaceutical Technology On-line, 25(2), 1-14 (2001). The use of chewing gum to achieve controlled release is described in WO 00/35298. The disclosures of these references are incorporated herein by reference in their entireties.

The compounds of the invention of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle-free injectors and infusion techniques. Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art. The solubility of compounds of the invention used in the preparation of parenteral solutions may be increased using appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus, compounds of the invention may be formulated as a solid, semi-solid, or thixotropic liquid for administration as an implanted depot providing modified release of the active compound. Examples of such formulations include drug-coated stents and PGLA microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated; see, for example, J Pharm Sci, 88 (10), 955-958 by Finnin and Morgan (October 1999). Other means of topical administration include delivery by electroporation, iontophoresis, phonophoresis, sonophoresis and micro needle or needle-free (e.g. Powderject™, Bioject™, etc.) injection. The disclosures of these references are incorporated herein by reference in their entireties. Formulations for topical administration may be formulated to be immediate and/or modified release. Modified release formulations include delayed-, sustained-, pulsed-, controlled-, targeted and programmed release.

The compounds of the invention of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (preferably an atomizer using electro-hydrodynamics to produce a fine mist), or nebulizer, with or without the use of a suitable propellant known within the art. For intranasal use, the powder may include a bio-adhesive agent, for example, chitosan or cyclodextrin.

The pressurized container, pump, spray, atomizer, or nebulizer contains a solution or suspension of the compound(s) of the invention comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid. Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

Capsules (made, for example, from gelatin or HPMC), blisters and cartridges for use in an inhaler or insufflator may be formulated to contain a powder mix of one or more compounds of the invention, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomizer using electrohydrodynamics to produce a fine mist may contain from 1 μg to 20 mg of a compound of the invention per actuation and the actuation volume may vary from 1 μL to 100 μL. A typical formulation includes one or more compounds of the invention, propylene glycol, sterile water, ethanol and sodium chloride. Alternative solvents which may be used instead of propylene glycol include glycerol and polyethylene glycol. Suitable flavors, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, poly(DL-lactic-coglycolic acid (PGLA). Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing, preferably, a desired amount of a compound of the invention The overall daily dose may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate. Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronized suspension or solution in isotonic, pH adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis. Formulations for ocular/aural administration may be formulated to be immediate and/or modified release. Modified release formulations include delayed-, sustained-, pulsed-, controlled-, targeted, or programmed release.

The compounds of the invention and suitable derivatives thereof or polyethylene glycol-containing polymers, in order to improve their solubility, dissolution rate, taste-masking, bioavailability and/or stability for use in any of the modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gamma-cyclodextrins, examples of which may be found in PCT Publication Nos. WO 91/11172, WO 94/02518 and WO 98/55148, the disclosures of which are incorporated herein by reference in their entireties.

The invention also relates to a pharmaceutical composition for the treatment of abnormal cell growth in a mammal, including a human, which comprises an amount of a cis-resveratrol or stabilized cis-resveratrol isomer, as defined above (including hydrates, solvates and polymorphs of said compound or pharmaceutically acceptable salts thereof), in combination with one or more (preferably one to three) additional anti-cancer agents. In particular embodiments, the one or more additional anti-cancer agents are targeted agents, such as inhibitors of Pl3 kinase, mTOR, PARP, IDO, TOO, ALK, ROS, MEK, VEGF, FL T3, AXL, ROR2, EGFR, FGFR, Src/Abl, RTK/Ras, Myc, Raf, PDGF, AKT, c-Kit, erbB, CDK2, CDK2/4/6, CDK4/6, CDK5, CDK7, CDK9, SMO, CXCR4, HER2, GLS1, EZH2 or Hsp90, or immunomodulatory agents, such as PD-1 or PD-L 1 antagonists, OX40 agonists or 4-1 BB agonists. In other embodiments, the one or more additional anti-cancer agents are standard of care agents, such as tamoxifen, docetaxel, paclitaxel, cisplatin, capecitabine, gemcitabine, vinorelbine, exemestane, letrozole, fulvestrant, anastrozole or trastuzumab.

In another embodiment, the invention provides a pharmaceutical composition comprising a cis-resveratrol or stabilized cis-resveratrol isomer or a pharmaceutically acceptable salt thereof, and a pharmaceutically acceptable carrier or excipient and a pharmaceutical composition comprising a cis-resveratrol or stabilized cis-resveratrol isomer or a pharmaceutically acceptable salt thereof, and a pharmaceutically acceptable carrier or excipient. In some embodiments, the pharmaceutical compositions comprise two or more pharmaceutically acceptable carriers and/or excipients. In other embodiments, the pharmaceutical composition further comprises at least one additional anti-cancer agent.

In some embodiments, a pharmaceutical composition of the invention further comprises at least one additional anti-cancer agent or a palliative agent. In some such embodiments, the at least one additional agent is an anti-cancer agent as described below. In some such embodiments, the combination provides an additive, greater than additive, or synergistic anti-cancer effect.

In one embodiment, the invention provides a method for the treatment of abnormal cell growth in a subject in need thereof, comprising administering to the subject a therapeutically effective amount of a pharmaceutical composition of the invention, or a pharmaceutically acceptable salt thereof.

In another aspect, the invention provides a method for the treatment of abnormal cell growth in a subject in need thereof, comprising administering to the subject an amount of a pharmaceutical composition of the invention, or a pharmaceutically acceptable salt thereof, in combination with an amount of an additional therapeutic agent (e.g., an anticancer therapeutic agent), which amounts are together effective in treating said abnormal cell growth.

In frequent embodiments of the methods provided herein, the abnormal cell growth is cancer. A pharmaceutical composition of the invention may be administered as single agents, for example a pharmaceutical composition of a cis-resveratrol or stabilized cis-resveratrol isomer, a pharmaceutical composition of a cis-resveratrol or stabilized cis-resveratrol isomer, or a pharmaceutical composition of a cis-resveratrol or stabilized cis-resveratrol isomer, or as a single pharmaceutical composition, or may be administered in combination with other anti-cancer agents, in particular standard of care agents appropriate for the particular cancer. In some embodiments, the methods provided result in one or more of the following effects: (1) inhibiting cancer cell proliferation; (2) inhibiting cancer cell invasiveness; (3) inducing apoptosis of cancer cells; (4) inhibiting cancer cell metastasis; or (5) inhibiting angiogenesis.

As used herein the singular forms “a”, “an”, and “the” include plural referents unless the context clearly dictates otherwise. Thus, for example, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Resveratrol Isomers Target Mitochondrial Endonuclease G to Antagonistically Modulate Elimination of Paternal Mitochondria and Neurodegeneration

Elimination of paternal mitochondria is a highly conserved developmental process in most animals and is important for normal development, organismal fitness, and proper tissue functions. In an effort to identify drugs that modulate PME, the present inventors conducted a candidate screen of compounds derived from natural products and found unexpectedly that resveratrol, a well-known natural product, could affect PME. Further analysis showed that resveratrol has two interconvertible isomers catalyzed by light absorption, and interestingly, that these two isomers exhibited opposite activities in PME, with trans-resveratrol enhancing and cis-resveratrol inhibiting PME. Chemical genetic analysis and biochemical analysis indicated that trans-resveratrol and cis-resveratrol target the paternal pathway, and specifically, the mitochondrial endonuclease G, CPS-6, to impact PME, through enhancing and suppressing the endonuclease activity of CPS-6, respectively. Therefore, resveratrol is a rare, isomer-dependent, dual-activity regulator of the mitochondrial endonuclease G, which plays important and diverse roles in apoptosis, neurodegeneration, PME, autophagy, and mitochondrial maintenance and functions through cleaving nuclear and mitochondrial DNA.

Resveratrol is a small molecule found in grapes, nuts and many plants and belongs to a group of polyphenolic compounds. Plant-derived resveratrol has been widely used as a nutritional supplement, because of its multiple proposed health benefits, including anti-oxidation, anti-cancer, anti-aging, anti-inflammation, and anti-virus properties, as well as cardioprotective and neuroprotective effects. However, despite the enormous interest in resveratrol and large number of related studies, how resveratrol works to confer those potential beneficial effects and whether it can truly deliver some of the beneficial effects remain largely unresolved. Moreover, results from numerous studies, including those from human clinical trials, are often conflicting, with some reporting toxicity and adverse side effects caused by the resveratrol treatment. For example, both the anti-aging effect of resveratrol, one of its highly touted health benefits, and its proposed in vivo target, NAD-dependent deacetylase sirtuin-1 (SIRT1), have been highly debated. Therefore, better understanding of the mechanisms of action of resveratrol and its physiological targets is vitally important.

The present inventors demonstrate compelling genetic, biochemical and cell biological evidence that resveratrol regulates elimination of paternal mitochondria during C. elegans development by targeting a crucial PME factor, the mitochondrial endonuclease G. More interestingly, the present inventors show that resveratrol has two readily interconverted isomers, trans-resveratrol and cis-resveratrol, which exhibit opposite activities to enhance and inhibit the endonuclease activity of EndoG in vitro and PME and α-synuclein-induced neurodegeneration in vivo, respectively. These results, in combination with the nanomolar affinity of resveratrol to EndoG, which is far higher affinity than other proposed resveratrol targets, establish EndoG as a new and one of the best in vivo targets of resveratrol. Our observations that aged trans-resveratrol was partially converted into cis-resveratrol and thus acquired an opposite activity in PME and that trans-resveratrol and cis-resveratrol are readily interconverted by light absorption suggest that extra care needs to be taken when analyzing the in vitro and in vivo impacts of resveratrol, which in most studies involves trans-resveratrol. The interconvertible nature of resveratrol isomers and their potential opposite effects on biological processes, as shown herein with PME and neurodegeneration, could contribute to some of the conflicting findings in resveratrol studies and the unexpected toxicity and adverse side effects caused by resveratrol treatments.

EndoG plays important roles in multiple different biological processes in diverse organisms, including apoptosis, paternal mitochondrial and mtDNA elimination during development, neurodegeneration, autophagy, mtDNA replication, and mitochondrial maintenance. Reduction or loss of EndoG has been shown to delay or block apoptosis, PME, autophagy, and neurodegeneration and can cause cardiac hypertrophy. The observations that resveratrol can prevent cancer and inflammation and have cardioprotective and neuroprotective activities could be related to the EndoG-enhancing or inhibitory activity of resveratrol isomers. Our findings that cis-resveratrol potently inhibits, and trans-resveratrol instead enhances, DA neuronal death in the C. elegans α-synuclein Parkinson's disease model through targeting EndoG provides the best evidence thus far that the neuroprotective activity of resveratrol involves regulation of the EndoG activity. These observations also underscore the importance of appropriate resveratrol treatments, which could benefit the treatment of patients, if applied with the appropriate resveratrol isomer, and could have deleterious outcomes, if the wrong resveratrol isomer is used. Therefore, trans-resveratrol and cis-resveratrol and their corresponding light-insensitive and more stable derivatives could be used selectively to treat different diseases, such as cancer, cardiac hypertrophy, inflammation, and neurodegenerative disease, when EndoG is involved in the development or pathogenesis of these diseases.

### Example 1: Screening for Compounds that Affect PME in C. elegans

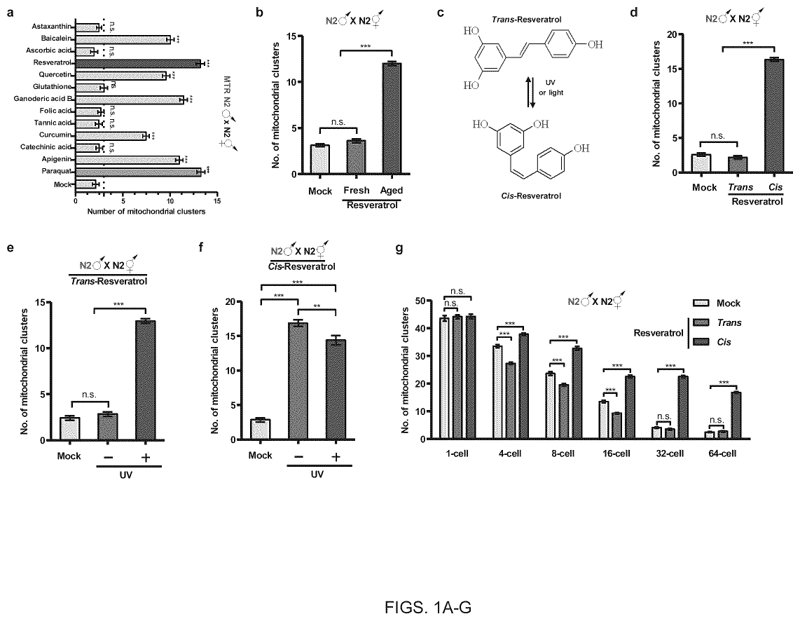

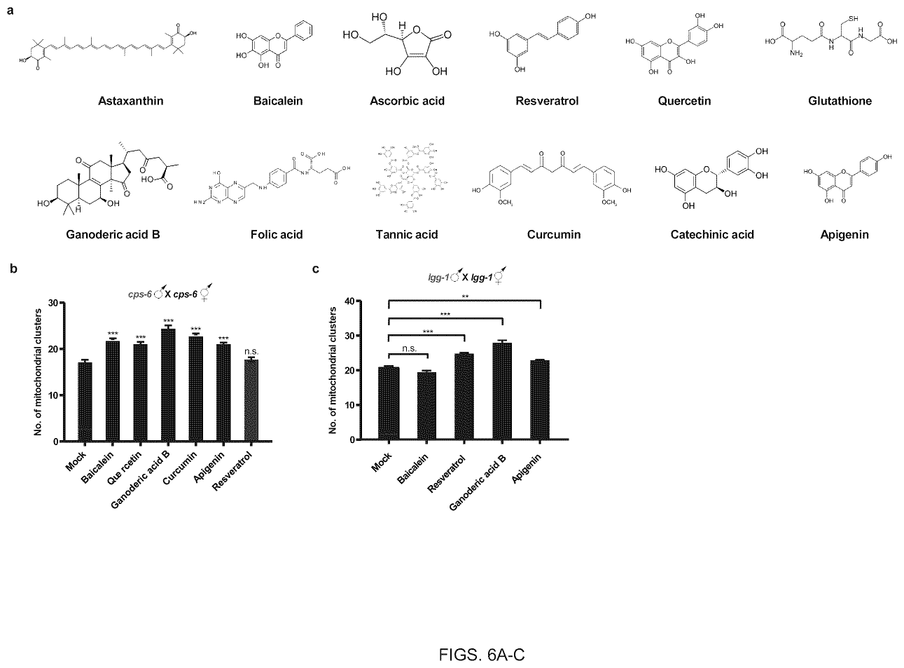

Following fertilization, paternal mitochondria are rapidly depolarized and damaged in fertilized eggs, which could generate and release reactive oxygen species (ROS) that affect PME and embryo development. We thus screened twelve small molecules derived from natural products that have been used as antioxidants for the potential activity in modulating PME (FIG. 6a), using a fluorescent microscopy assay. In this assay, C. elegans wild-type (N2) males prestained with Mitotracker Red (MTR), a mitochondrion-specific fluorescent dye, were mated with unstained N2 hermaphrodites in the presence of the compound of interest (100 PM) and the cross-fertilized embryos were examined for the presence of MTR-stained paternal mitochondria or mitochondrial clusters. In 64-cell stage embryos from mated animals treated with the dimethyl sulfoxide solvent (DMSO) mock control, an average of two MTR-stained paternal mitochondrial clusters were observed (FIG. 1a), which is comparable to that observed in the same mating experiment without drug treatment and indicates that paternal mitochondria are efficiently eliminated in fertilized eggs. Interestingly, treatment with six compounds, baicalein, resveratrol, quercetin, ganoderic acid B, curcumin, and apigenin, resulted in persistence of more paternal mitochondrial clusters (ranging from 7 to 13 clusters) in 64-cell stage embryos (FIG. 1a), suggesting that PME was delayed or partially inhibited. In comparison, treatment with the other six compounds (astaxanthin, ascorbic acid, glutathione, folic acid, tannic acid, and catechinic acid) did not inhibit PME (FIG. 1a). As a control, we performed the same experiment with paraquat, a known oxidant, and found that paraquat inhibited PME (FIG. 1a). Because some of the antioxidants do not affect PME and the oxidant, paraquat, does, the antioxidation property of the six compounds is unlikely the cause for the observed PME inhibitory activity.

### Example 2: Resveratrol Targets the CPS-6 Pathway to Affect PME

To investigate how these six compounds might affect PME, the present inventors performed chemical genetic analysis to analyze the PME pathways that might be affected by the compounds. The present inventors first examined whether they might affect the paternal PME pathway mediated by the mitochondrial endonuclease CPS-6, whose activity is inhibited by oxidation and protected by reducing agents. When MTR-stained males carrying a strong loss-of-function deletion, cps-6(tm3222), were mated with cps-6(tm3222) hermaphrodites in the presence of the DMSO control, an average of 17 MTR-stained paternal mitochondria clusters were observed in 64-cell stage embryos (FIG. 6b), confirming that CPS-6 is crucial for PEM. When the same mating experiments were conducted in the presence of one of the six compounds, five of them (baicalein, quercetin, ganoderic acid B, curcumin, and apigenin) significantly increased the number of paternal mitochondrial clusters in 64-cell cross-fertilized embryos (FIG. 6b), indicating that treatment with these five compounds enhanced the PME defect caused by loss of cps-6 and that these five compounds affect a pathway different from the CPS-6 PME pathway. Interestingly, treatment with resveratrol did not affect the PME defect of the cps-6(tm3222) embryos (FIG. 6b), suggesting that resveratrol likely targets the CPS-6 PME pathway. On the other hand, treatment with resveratrol, ganoderic acid B and apigenin, but not baicalein, enhanced the PME defect in cross-fertilized embryos deficient in the Igg-1 gene (FIG. 6c), which is required for autophagy in C. elegans and mediates the maternal autophagy pathway to promote PME. These results are consistent with resveratrol targeting the CPS-6 pathway to inhibit PME, while baicalein targets the autophagy pathway to affect PME.

### Example 3. Two Isomers of Resveratrol Exhibit Opposite Activities in PME

It is surprising that resveratrol, a well-known antioxidant, shows an activity in suppressing PME mediated by CPS-6, whose nuclease activity is diminished by oxidation and enhanced by antioxidants. Because the resveratrol used in the drug screen had been used multiple times (termed aged resveratrol) and may have been modified or converted into a different chemical in the air over time, we tested if freshly prepared resveratrol solution affected PME. We found that fresh resveratrol did not inhibit PME (FIG. 1b), whereas the aged resveratrol solution maintained the PME inhibitory activity. These results indicate that the aged resveratrol may have acquired a new PME regulatory activity. Resveratrol has two isomeric forms, cis-resveratrol and trans-resveratrol, which can be interconverted by light absorption (FIG. 1c). Trans-resveratrol is found in fruits and plants and thought to have various beneficial health effects, such as antioxidation, anti-cancer, and neuroprotection. In our initial drug screen, trans-resveratrol was used. However, only the aged trans-resveratrol solution, not the freshly prepared trans-resveratrol solution, showed PME inhibitory activity, suggesting that trans-resveratrol in the aged solution may have been converted into a different chemical, possibly cis-resveratrol (FIG. 1b). To test this possibility, we treated wild-type mating animals with freshly prepared trans-resveratrol and cis-resveratrol, respectively, and found that fresh cis-resveratrol inhibited PME better than the aged trans-resveratrol (FIG. 1d), generating an average of 16 paternal mitochondrial clusters in 64-cell embryos. In contrast, fresh trans-resveratrol did not inhibit PME (FIG. 1d). Moreover, when we used the ultraviolet (UV) to irradiate the fresh trans-resveratrol solution for 4 hours and then used the irradiated solution to treat wild-type mating animals, the irradiated trans-resveratrol acquired the activity to inhibit PME (FIG. 1e), almost as well as cis-resveratrol and the aged trans-resveratrol (FIG. 1b, d). These results suggest that cis-resveratrol can inhibit PME and trans-resveratrol cannot, but can acquire such an activity through UV-induced conversion into cis-resveratrol.

### Example 4: Interconversion of Two Resveratrol Isomers and their Impact on PME During Development

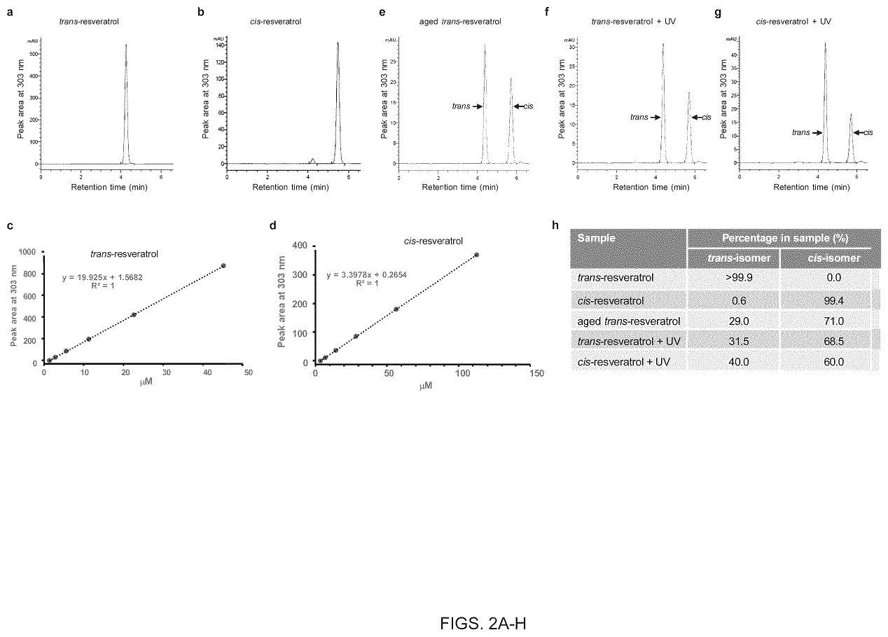

To confirm the interconversion between trans-resveratrol and cis-resveratrol and to quantitatively measure the ratio of cis- and trans-isomers in the resveratrol solution, we performed high performance liquid chromatography (HPLC) analysis, which can effectively separate trans- and cis-isomers (FIG. 2a, b). Because of the difference in extinction coefficients between cis-resveratrol and trans-resveratrol, and in order to determine absolute ratios of cis and trans isomers in resveratrol samples, calibration curves from 0 to 45 μM trans-resveratrol and 0 to 110 μM cis-resveratrol were obtained, respectively, which showed a linear correlation between the peak areas measured at the wavelength of 303 nm and the concentrations of the resveratrol isomers [y=19.9x+1.57 with r²=1 for the trans isomer and y=3.40x+0.27 with r²=1 for the cis isomer; FIG. 2c,d). We found that the aged trans-resveratrol solution used for the drug screen contained 29% of trans-resveratrol and 71% of cis-resveratrol (FIG. 2e,h), indicating that most of the trans-resveratrol in the solution had converted into the cis-resveratrol. This ratio of trans- and cis-resveratrol accounts for the unexpected PME inhibition by the aged trans-resveratrol solution (FIG. 1a, b), because cis-resveratrol could inhibit PME (FIG. 1d) and became the dominant isomer in this aged trans-resveratrol solution (FIG. 2h). Similarly, when pure trans-resveratrol was exposed to UV, 68.5% of trans-resveratrol were converted into cis-resveratrol (FIG. 2f,h) and thus acquired PME inhibitory activity after UV irradiation (FIG. 1e). On the other hand, when pure cis-resveratrol was exposed to UV, 40% of cis-resveratrol was converted into trans-resveratrol (FIG. 2g,h) and the irradiated cis-resveratrol solution exhibited reduced PME inhibitory activity (FIG. 1f), probably because cis-resveratrol was still the dominant isomer (60%) in the mixture. These results confirm that both isomers can be interconverted by UV.

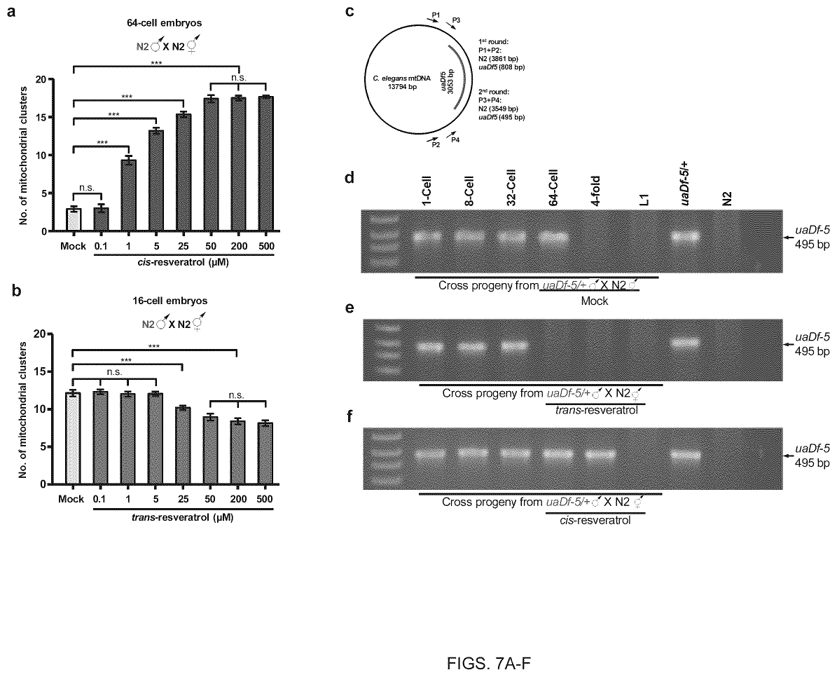

To further analyze the effects of resveratrol isomers on PME during embryo development, we monitored the dynamics of PME at different stages of cross-fertilized embryos treated with 0.25% ethanol (mock), trans-resveratrol, or cis-resveratrol. Compared with the mock control, cis-resveratrol blocked rapid elimination of paternal mitochondria during embryo development (FIG. 1g), resulting in persistence and significantly higher numbers of paternal mitochondria in all embryonic stages examined, including 32-cell and 64-cell stage embryos, when virtually all paternal mitochondria had been eliminated at the same stages of mock-treated embryos (FIG. 1g). Interestingly, in 4-cell, 8-cell and 16-cell embryos treated with trans-resveratrol, significantly less paternal mitochondria were observed than those in same stages of mock-treated embryos (FIG. 1g), suggesting that trans-resveratrol accelerated elimination of paternal mitochondria. In PME assays with different concentrations of trans-resveratrol and cis-resveratrol, 1 μM of cis-resveratrol was sufficient to significantly inhibit PME, with 50 μM of cis-resveratrol showing maximal PME inhibitory activity (FIG. 7a). On the other hand, trans-resveratrol could significantly enhance PME at 25 μM and reached maximal PME enhancing activity at 50 μM (FIG. 7b). These results suggest that cis-resveratrol is more potent in affecting PME, and more surprisingly, that cis-resveratrol and trans-resveratrol have opposite activities in regulating PME.

We confirmed these findings using a polymerase chain reaction (PCR)-based PME assay, which monitors the fate of paternal mtDNA in fertilized eggs from males carrying a 3053 bp mtDNA deletion allele (uaDf5) in a heteroplasmic mixture with wild-type mtDNA (FIG. 7c). In mating between N2 hermaphrodites and uaDf5/+ males treated with the mock control, PCR products derived specifically from paternal uaDf5 mtDNA were detected only in early stage cross-fertilized embryos, up to 64-cell stage embryos (FIG. 2d). This result is consistent with the findings from the same mating experiment without the mock treatment and with the results obtained from the fluorescent microscopy assay (FIG. 1g). Treatment with 50 μM of trans-resveratrol resulted in accelerated degradation of uaDf5 mtDNA (FIG. 2e), which disappeared from 64-cell stage or older embryos, whereas treatment with 50 μM of cis-resveratrol caused persistence of uaDf5 mtDNA to the late 4-fold embryos with around 600 cells. These results confirm that trans-resveratrol enhances and cis-resveratrol suppresses PME.

### Example 5: Resveratrol Isomers Act Paternally Through the CPS-6 Pathway to Affect PME

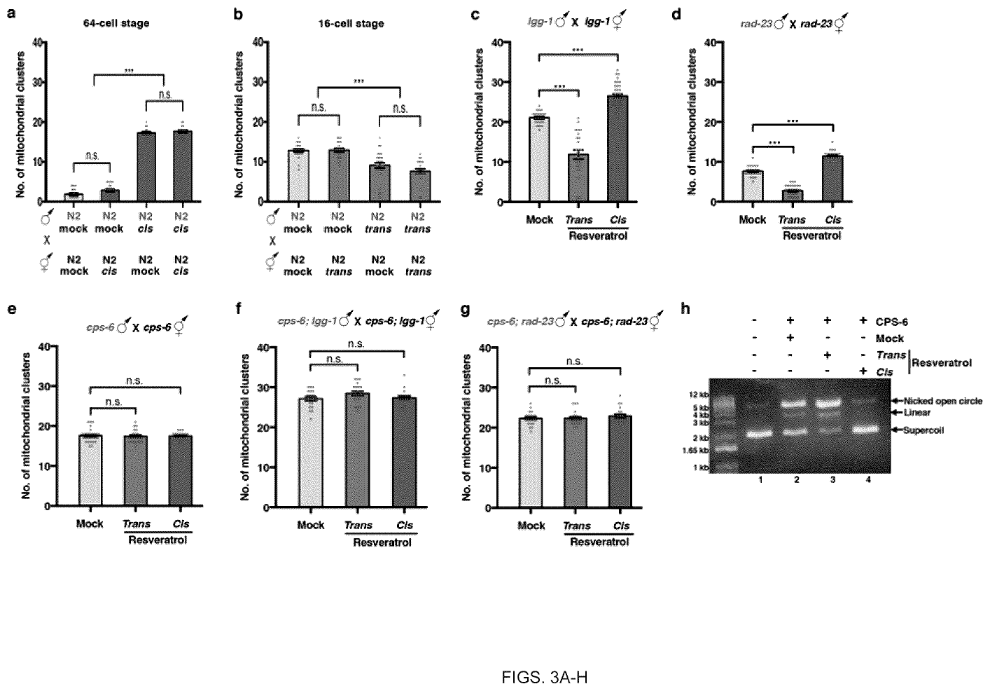

We next examined where two resveratrol isomers act to affect PME. MTR-stained N2 males treated with 0.25% ethanol (mock) or 50 μM of cis-resveratrol were mated with N2 hermaphrodites treated with mock or 50 μM of cis-resveratrol. 64-cell cross-fertilized embryos were examined for the presence of paternal mitochondrial clusters. In embryos derived from males treated with cis-resveratrol, PME was suppressed regardless of the resveratrol treatment status of hermaphrodites (FIG. 3a). In contrast, in embryos derived from males with the mock treatment, PME proceeded normally even when hermaphrodites were treated with cis-resveratrol (FIG. 3a). These results suggest that cis-resveratrol acts on a target in males or acts paternally, not maternally, to inhibit PME. We obtained similar results with trans-resveratrol (FIG. 3b), which only acted through males to enhance PME. Therefore, both cis-resveratrol and trans-resveratrol act paternally to affect PME, which is consistent with the observation that resveratrol specifically targets the CPS-6 pathway (FIG. 6b), which functions paternally to promote PME.

We also examined if resveratrol might affect two other PME pathways, the autophagy and the proteasome pathways. We found that trans-resveratrol suppressed and cis-resveratrol enhanced the PME defects of the Igg-1(bp523) mutant (FIG. 3c), which is defective in autophagy. Similarly, trans-resveratrol inhibited and cis-resveratrol enhanced the PME defects in animals deficient in the rad-23 gene (FIG. 3d), which encodes a ubiquitin receptor important for proteasomal degradation and acts maternally to promote PME. However, neither cis-resveratrol nor trans-resveratrol could enhance or suppress the PME defect in the cps-6(tm3222) mutant (FIG. 3e) or in the cps-6(tm3222); Igg-1(bp523) and cps-6(tm3222); rad-23(tm2595) double mutants (FIG. 3f,g), confirming that both resveratrol isomers target components in the CPS-6 pathway to affect PME.

### Example 6: Trans-Resveratrol Enhances and Cis-Resveratrol Inhibits the Nuclease Activity of CPS-6

Because resveratrol is an antioxidant and the endonuclease activity of CPS-6 is regulated by redox conditions both in vitro and in vivo, we investigated the possibility that resveratrol isomers could directly affect the nuclease activity of CPS-6 using a plasmid DNA cleavage assay. Recombinant CPS-6 protein was incubated with the supercoiled plasmid DNA substrate in the presence or absence of 20 μM trans-resveratrol or cis-resveratrol and the reactions were resolved by an agarose gel. Incubation with CPS-6 alone resulted in single-strand nicking and double-strand breaks of the plasmid DNA and the mobility shift of the plasmid DNA from the supercoiled form to the predominantly nicked open circle form and some linear form (FIG. 3h, lane 2), respectively. In the presence of trans-resveratrol, more supercoiled plasmid DNA was converted into the nicked open circle form and the linear form (FIG. 3h, lane 3), suggesting that trans-resveratrol enhances the endonuclease activity of CPS-6. In contrast, incubation with cis-resveratrol caused inhibition of both nicking and double-strand cleavage of the plasmid DNA by CPS-6 and the disappearance of the nicked open circle form and the linear form (FIG. 3h, lane 4), suggesting that cis-resveratrol inhibits CPS-6 nuclease activity. Since the nuclease activity of CPS-6 is required for PME, these in vitro results are consistent with the in vivo observations that trans-resveratrol enhances and cis-resveratrol inhibits PME. They also suggest that resveratrol isomers directly target the CPS-6 nuclease to impact PME.

### Example 7: Trans-Resveratrol Enhances and Cis-Resveratrol Inhibits Autophagosome Formation During PME

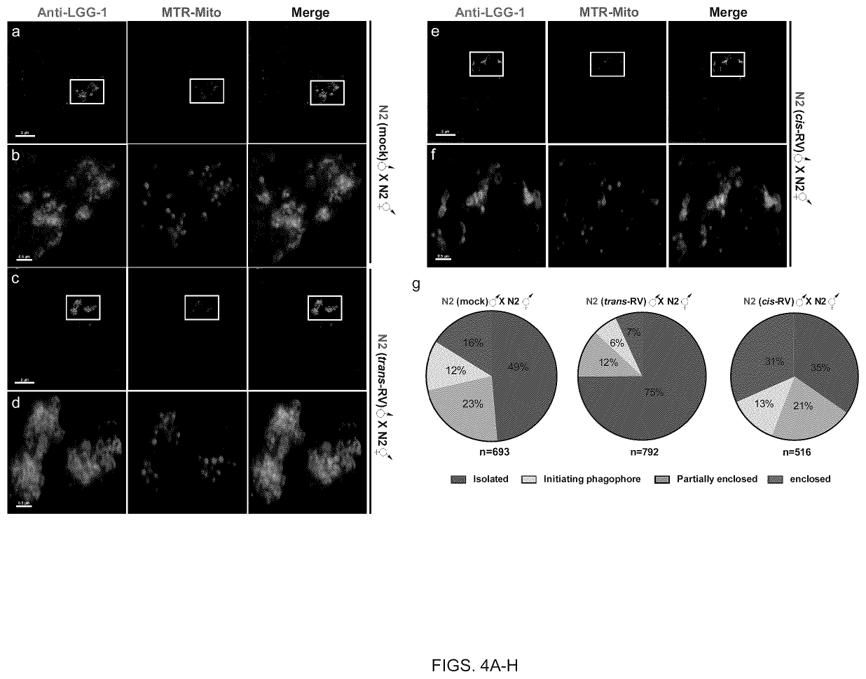

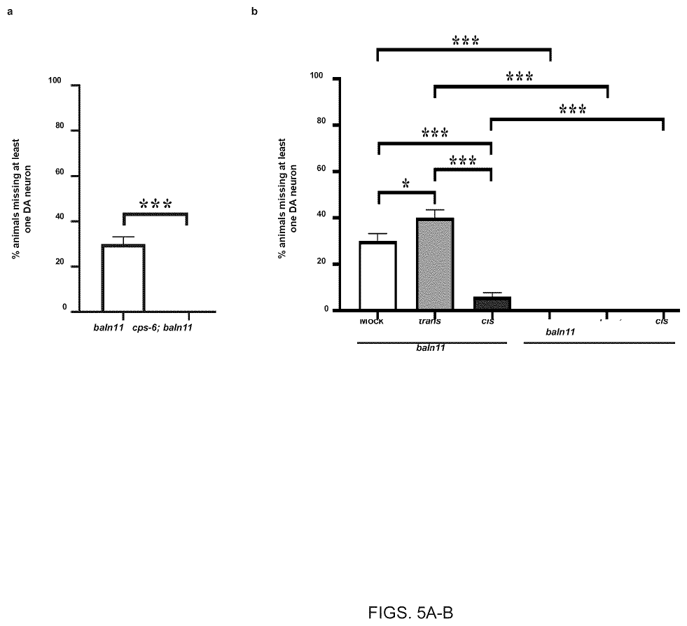

CPS-6 plays a key role in promoting internal breakdown of paternal mitochondria and rapid autophagosome formation on damaged paternal mitochondria following fertilization, which leads to their subsequent degradation by autophagy. We tested if resveratrol isomers affect autophagosome formation on paternal mitochondria using superresolution structured illumination microscopy (SIM). In zygotes from mating of MTR-stained and mock-treated N2 males with unstained N2 hermaphrodites, close to one half (49%) of MTR-stained paternal mitochondria were fully enclosed by autophagosomes labeled by anti-LGG-1 monoclonal antibody (FIG. 4a, b and g). The remaining paternal mitochondria either were partially enclosed by autophagosomes (23%), or had adjacent autophagosome membrane that could initiate the elongation process along the paternal mitochondrion (12%; initiating phagophore), or were isolated (16%) with no nearby autophagosome or phagophore (FIG. 4a, b and g). In zygotes from mating of MTR-stained and trans-resveratrol-treated N2 males with N2 hermaphrodites, autophagosome formation on paternal mitochondria increased dramatically, with 75% of paternal mitochondria fully enclosed and 12% of paternal mitochondria partially enclosed by autophagosomes (FIG. 4c, d and g). Only 7% of paternal mitochondria remained isolated, indicating that autophagosome formation on paternal mitochondria was substantially improved by treatment with trans-resveratrol. On the other hand, in zygotes from mating between unstained N2 hermaphrodites and MTR-stained and cis-resveratrol-treated N2 males, autophagosome formation on paternal mitochondria decreased substantially. Only 35% of paternal mitochondria were fully enclosed by autophagosomes, whereas the number of isolated paternal mitochondria increased substantially to 31% (FIG. 4e, f and g), suggesting that cis-resveratrol treatment inhibits autophagosome formation on paternal mitochondria. Again, these results suggest that trans-resveratrol enhances and cis-resveratrol inhibits the activity of CPS-6 and CPS-6-mediated autophagosome formation on paternal mitochondria.

### Example 8: Trans-Resveratrol and Stabilized Trans-Resveratrol Enhances and Cis-Resveratrol Inhibits α-Synuclein-Induced Neurodegeneration

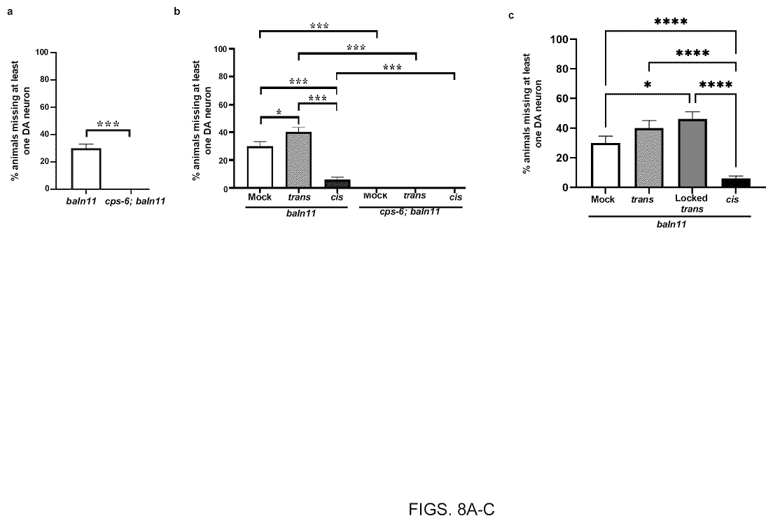

In addition to PME and apoptosis, EndoG was previously shown to play an important role in mediating dopaminergic (DA) neuronal death induced by expression of human α-synuclein, which is widely considered to be a major proponent of Parkinson's disease. However, the mechanism by which EndoG mediates α-synuclein-induced neurodegeneration is unclear. In a C. elegans α-synuclein-based Parkinson's Disease model, co-expression of GFP and human α-synuclein in C. elegans dopaminergic neurons under the control of the promoter of the dat-1 gene, which encodes a dopamine transporter, from an integrated transgene baIn11 (Pdat-1::gfp/Pdat-1::α-synuclein) caused DA neuronal death in 30% of adult baIn11 animals (FIG. 8a). The cps-6(tm3222) mutation blocked DA neuronal death in adult baIn11 animals (FIG. 8a), confirming that CPS-6 is important for α-synuclein-induced DA neuronal death. The present inventors then tested whether resveratrol isomers might affect DA neuronal death in baIn11 animals and found that cis-resveratrol strongly inhibited DA neuronal death (FIG. 8b), protecting most adult baIn11 animals (94%) from neurodegeneration. In contrast, trans-resveratrol enhanced dopaminergic neuron loss, causing neurodegeneration in 40% of adult baIn11 animals, compared with 30% of animals with DA neuronal loss in mock treatment. Stabilized (also referred to herein as “locked”) trans-resveratrol similarly enhanced DA neuron loss, causing neurodegeneration in 45% of adult baIn11 animals, slightly higher than that caused by trans-resveratrol treatment (FIG. 8c). Importantly, resveratrol isomers did not enhance or alter DA neuronal death in cps-6(tm3222); baIn11 animals (FIG. 8b). These results are consistent with cis-resveratrol inhibiting and trans-resveratrol or locked trans-resveratrol enhancing DA neuronal death through targeting the CPS-6 endonuclease, which is crucial to drive α-synuclein-induced dopaminergic neurodegeneration.

### Example 9: Cis-Resveratrol Inhibits and Trans-Resveratrol Enhances Tumor Growth

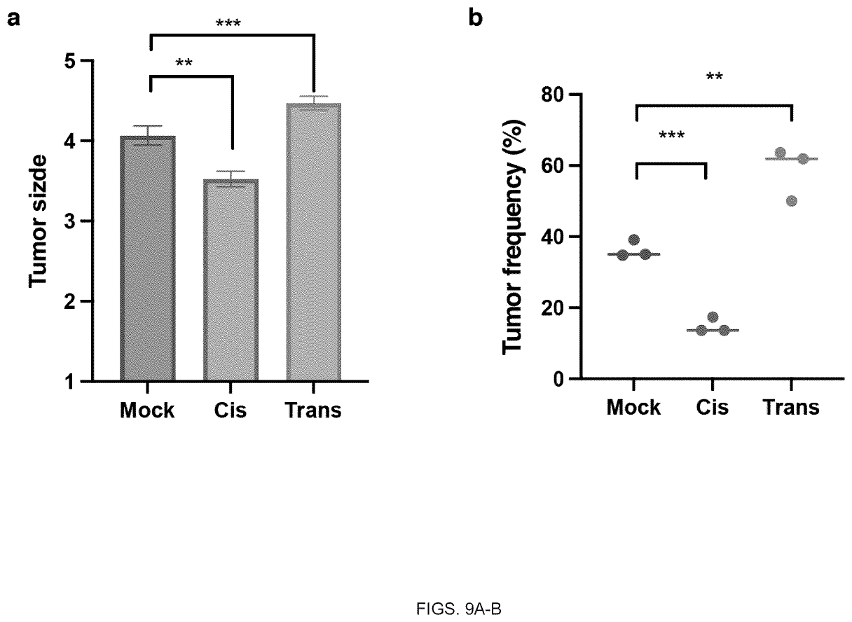

The present inventors tested whether resveratrol isomers affect tumorigenesis in a C. elegans germline tumor model (ET507), in which three cell growth genes, daf-16, cki-2, and glp-1, are mutated, leading to uncontrolled germ cell proliferation and germline tumor. We found that cis-resveratrol treatment reduces both the tumor size and the tumor occurring frequency in ET507 animals (FIG. 9), suggesting that cis-resveratrol inhibits tumor growth. On the other hand, trans-resveratrol treatment increases the size of tumors and their occurring frequency in ET507 animals (FIG. 9), indicating that trans-resveratrol promotes tumor formation. These results indicate that trans-resveratrol and cis-resveratrol again have opposite functions in promoting and inhibiting tumor formation and that cis-resveratrol or its stabilized derivative can be used to treat cancer.

### Example 10: Trans-Resveratrol Prevents Embryonic Lethality Caused by Persistent Paternal Mitochondria

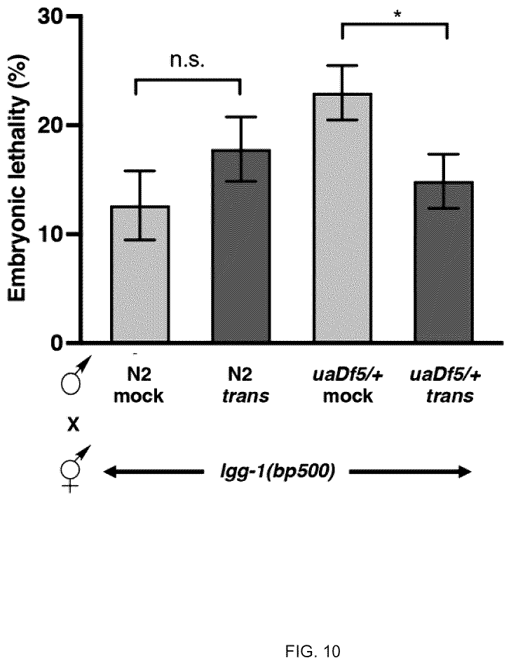

Several studies have shown that abnormal persistence of paternal mitochondria is detrimental to normal development and causes various deficiencies, including neurological and muscular defects, which lack treatment options. For example, when uaDf5 mutant paternal mitochondria abnormally persisted due to loss of the maternal autophagy gene, lgg-1, the persistent uaDf3 paternal mitochondria caused a significantly higher rate of embryonic lethality (FIG. 10), compared with embryos with persistent wild-type paternal mitochondria. Treatment with 100 μM trans-resveratrol, which enhances removal of paternal mitochondria, significantly reduced embryonic lethality caused by uaDf5 paternal mitochondria (FIG. 10). These results provide strong evidence that trans-resveratrol or its stabilized derivative can be used to treat disorders caused by persistent paternal mitochondria or damaged mitochondria in general.

### Example 11: Stabilized Trans-Resveratrol Enhances the Nuclease Activity of CPS-6 Like Trans-Resveratrol and is UV Resistant

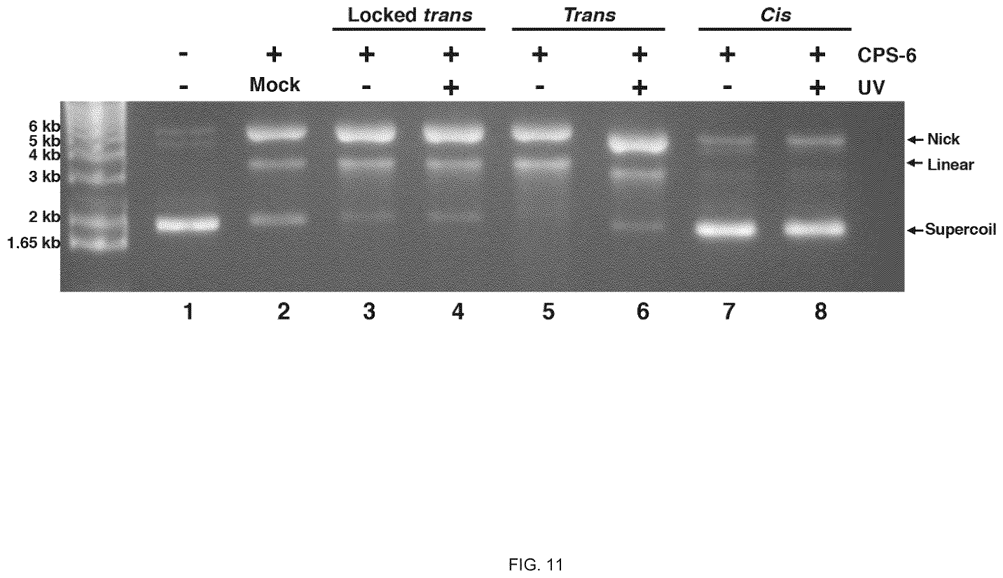

The present inventors tested whether stabilized (sometimes referred to as “locked”) trans-resveratrol affects the nuclease activity of CPS-6 using the plasmid cleavage assay. Incubation of stabilized trans-resveratrol with CPS-6 resulted in conversion of more supercoiled plasmid DNA into the nicked open circle form and the linear form (FIG. 11, lane 3), suggesting that stabilized trans-resveratrol similarly enhance the endonuclease activity of CPS-6 like trans-resveratrol (FIG. 11, lane 5). Interestingly, after irradiated by UV for 3 hours, trans-resveratrol lost its activity to enhance the nuclease activity of CPS-6, producing less nicked open circle form and the linear form of DNA (FIG. 11, lane 6), showing a plasmid DNA cleavage pattern similar to that of mock-treated CPS-6 (FIG. 11, lane 2). This result suggests that some of the trans-resveratrol was converted into cis-resveratrol, which is a CPS-6 inhibitor (FIG. 11, lane 7). In contrast, 3-hour UV irradiation did not alter the activity of stabilized trans-resveratrol in enhancing CPS-6 nuclease activity to cleave plasmid DNA (FIG. 11, lanes 3 and 4), indicating that stabilized trans-resveratrol is resistant to UV irradiation and is a light-insensitive, stable trans-resveratrol derivative that could be superior to trans-resveratrol in disease treatment.

### Example 12: Stabilized Trans-Resveratrol Enhances PME as Well as Trans-Resveratrol

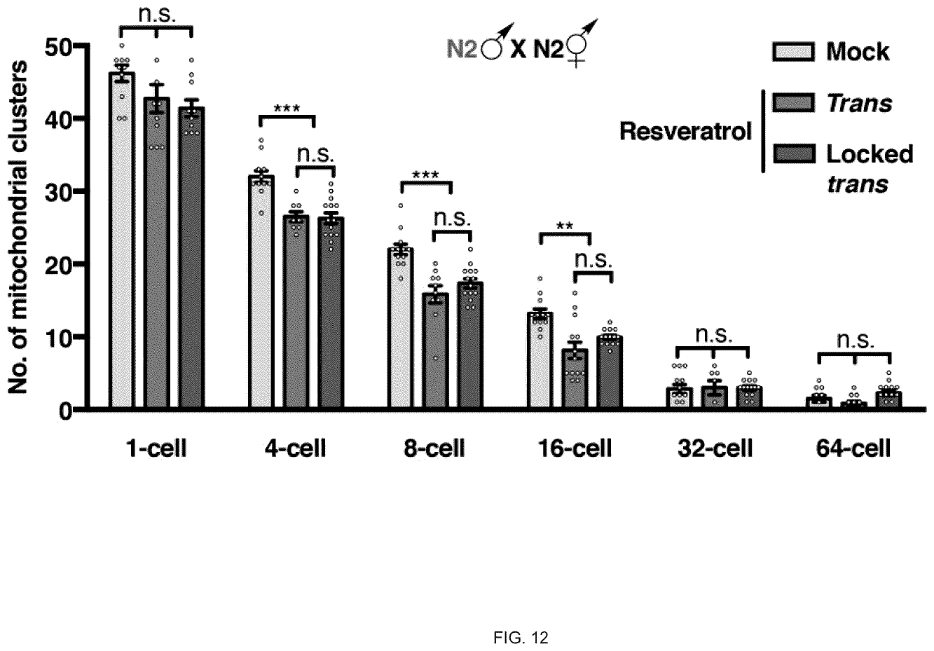

The present investors further tested whether stabilized trans-resveratrol affects PME by monitoring the dynamics of paternal mitochondrial elimination at different stages of cross-fertilized embryos between MTR-stained N2 males and N2 hermaphrodites treated with 0.25% ethanol (mock), 50 μM stabilized trans-resveratrol, or 50 μM trans-resveratrol. We found that stabilized trans-resveratrol and trans-resveratrol showed identical activity in promoting PME, accelerating elimination of paternal mitochondria in 4-cell, 8-cell and 16-cell embryos, compared with same stages of mock-treated embryos (FIG. 12). These results suggest that stabilized trans-resveratrol acts like trans-resveratrol in vivo to promote removal of damaged mitochondria and potentially can be used to treat diseases caused by persistent paternal mitochondria or persistent damaged mitochondria in general.

### Example 13: Synthesis of a Stabilized Trans- and Cis-Resveratrol Isomer

The synthesis of a locked aza-derivative trans-resveratrol is described by Fujita Y, et al. Aza-derivatives of resveratrol are potent macrophage migration inhibitory factor inhibitors. Invest New Drugs. 2012 October; 30(5):1878-86, and Luo Y., et al. cis-trans Isomerisation of substituted aromatic imines: a comparative experimental and theoretical study. Chenphyschem. 2011 Aug. 22; 12(12):2311-21. The methods of synthesizing and isolating stabilized trans-resveratrol having an aza-substitution as described by Fujita and Luo are specifically incorporated herein by reference.

The present inventors have provided a step-wise synthesis for the production of a novel resveratrol derivative stabilized in the cis-configuration by a 5-member aromatic ring having a ketone group (2-Cyclopentanone). In this embodiment, the stabilized cis-resveratrol isomer compound according to Formula IV:

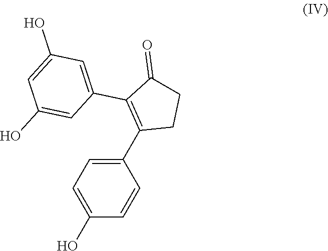 [SMILES: O=C1CCC(c2ccc(O)cc2)=C1c1cc(O)cc(O)c1]

As shown in Scheme 1 below, to a stirred solution of 3-(4-methoxyphenyl)cyclopent-2-en-1-one (2) (1.0 g, 5.32 mmol) and pyridine-N-oxide (5.85 mmol) in CH3CN (30 mL) was added NBS (6.38 mmol) at 0° C. The stirring continued at the same temperature for 1 h. The reaction mixture was quenched with water (30 mL) and extracted with DCM (3×50 mL). The combined organic layer was washed with brine (30 mL), dried over anhydrous Na2SO4, filtered, and evaporated under reduced pressure. The crude product was purified by flash chromatography on silica gel (AcOEt/cyclohexane, 1:9)

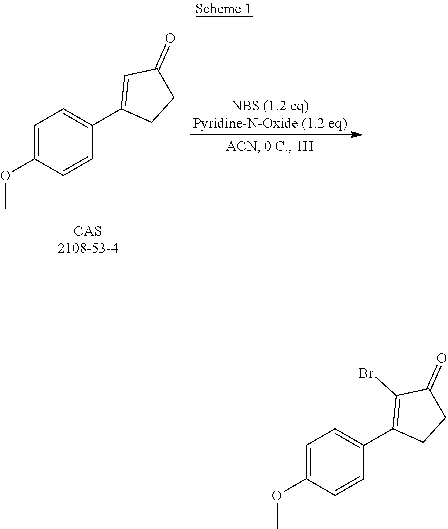

| Reagent | Equiv | mw | g | mmoles |
|---|---|---|---|---|
| 3-(4-methoxyphenyl) | 1 | 188.22 | 1.00 | 5.31 |
| cyclopent-2-en-1 [symbol] | | | | |
| NBS | 1.2 | 177.98 | 1.13 | 6.38 |
| Pyridine-N-Oxide | 1.2 | 95.10 | 0.61 | 6.38 |
| Solvents | mL/gram | mL | | |
| ACN | 30 | 30 | | |

[symbol]

indicates data missing or illegible when filed

As shown in Scheme 2 below, to a stirred solution of 2-bromo-3-(4-methoxyphenyl) cyclopent-2-en-1-one (3) (1.0 g, 3.75 mmol) in toluene and water (8:2) mixed solvents (10 mL) were added (2-benzyloxyphenyl)-boronic acid (5.62 mmol) and Na2CO3 (11.25 mmol). The mixture was degassed with nitrogen for 10 min followed by the addition of Pd(PPh3)4 (0.187 mmol) and stirred at 100° C. for 14 h. The reaction mixture was cooled to an ambient temperature, diluted with water (30 mL), and extracted with AcOEt (3×50 mL). The combined organic layer was washed with brine (1×30 mL), dried over anhydrous Na2SO4, and filtered, and the volatiles were evaporated. The crude was purified by flash chromatography on silica gel (AcOEt/cyclohexane, 1:4) to get 4 as a white solid.

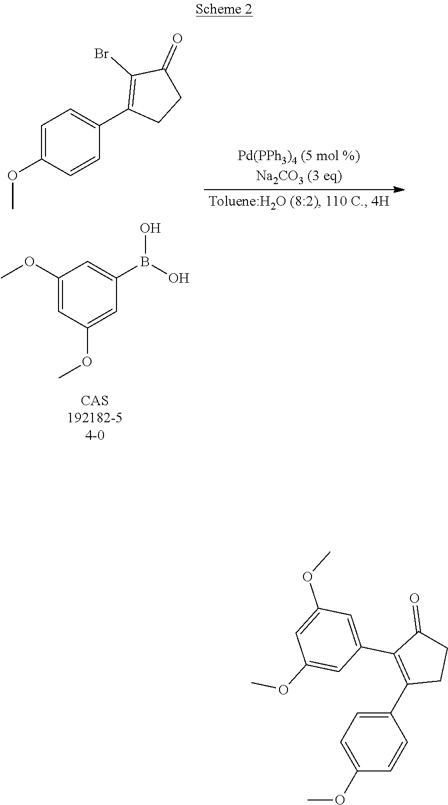 [SMILES: COc1ccc(C2=C(Br)C(=O)CC2)cc1 ; COc1cc(OC)cc(B(O)O)c1 ; COc1ccc(C2=C(c3cc(OC)cc(OC)c3)C(=O)CC2)cc1]

| Reagent | Equiv | mw | g | mmoles |
|---|---|---|---|---|
| 2-bromo-3-(4-methoxyphenyl) | 1 | 267.118 | 1.00 | 3.74 |
| cyclopent-2-en-1- [symbol] | | | | |
| 3,5 Dimethoxyphenylboronic | 1.5 | 181.98 | 1.02 | 5.62 |
| acid | | | | |
| Pd(PH3)4 | 0.05 | 1155.56 | 0.22 | 0.19 |
| Na2CO3 | 3 | 105.99 | 1.19 | 11.23 |
| Solvents | mL/gram | mL | | |
| Tolune:Water (8:2) | 10 | 10 | | |

[symbol]

indicates data missing or illegible when filed

As shown in Scheme 2 below, To a solution of 2-(3,5-dimethoxyphenyl)-3-(4-methoxyphenyl)cyclopent-2-en-1-one (1.00 g, 3.08 mmol) in CH₂Cl₂ (10 mL) was added dropwise 11.10 mL of BBr3 in CH₂Cl₂ (1.0 M, 11.1 mmol) under nitrogen at −78° C. The reaction mixture was allowed to warm to room temperature and further stirred at room temperature for 24 h. After ice water was added, the solution was extracted with ethyl acetate. The organic layer was washed with water and then dried over Na2SO4.

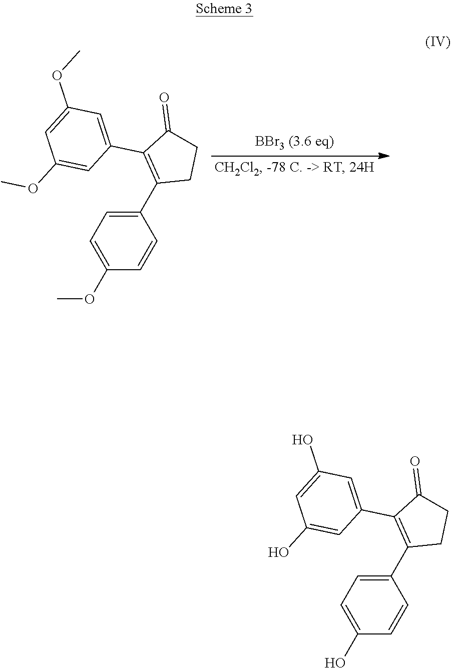 [SMILES: COc1ccc(C2=C(c3cc(OC)cc(OC)c3)C(=O)CC2)cc1 ; O=C1CCC(c2ccc(O)cc2)=C1c1cc(O)cc(O)c1]

### Example 14: Comparative Activity of Stabilized Cis-Resveratrol and Cis-Resveratrol

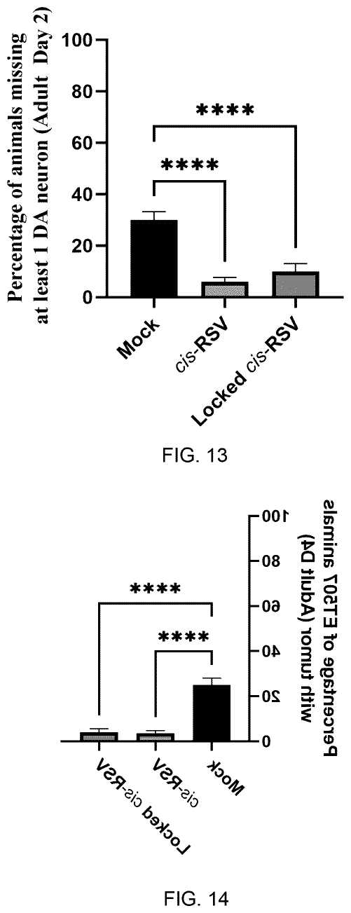

Stabilized cis-resveratrol shows a comparable activity as cis-resveratrol in suppressing α-synuclein-induced dopaminergic neuronal death and germline tumor formation. As described above, Applicant's successfully synthesized a new compound, stabilized cis-resveratrol. In both neuronal death and tumor assays, stabilized cis-resveratrol shows a comparable activity as cis-resveratrol in strongly suppressing α-synuclein-induced dopaminergic neuronal death (FIG. 13) and germline tumor formation (FIG. 14). No obvious toxicity was observed with stabilized cis-resveratrol in our animal assays. These results demonstrate that the cis-resveratrol locking process does not interfere with its therapeutic efficacy.

### REFERENCES

1. Calvo, S. E. & Mootha, V. K. The mitochondrial proteome and human disease. Annu Rev Genomics Hum Genet 11, 25-44 (2010).
2. Wang, X. The expanding role of mitochondria in apoptosis. Genes Dev 15, 2922-2933 (2001).
3. Cummins, J. M. Fertilization and elimination of the paternal mitochondrial genome. Human Reproduction 15, 92-101 (2000).
4. Wallace, D. C. Why Do We Still Have a Maternally Inherited Mitochondrial DNA? Insights from Evolutionary Medicine. Annual Review of Biochemistry 76, 781-821 (2007).
5. Zhou, Q. et al. Mitochondrial endonuclease G mediates breakdown of paternal mitochondria upon fertilization. Science 353, 394-399 (2016).
6. Zhou, Q., Li, H. & Xue, D. Elimination of paternal mitochondria through the lysosomal degradation pathway in C. elegans. Cell Res 21, 1662-1669 (2011).
7. Al Rawi, S. et al. Postfertilization autophagy of sperm organelles prevents paternal mitochondrial DNA transmission. Science 334, 1144-1147 (2011).
8. Sato, M. & Sato, K. Degradation of paternal mitochondria by fertilization-triggered autophagy in C. elegans embryos. Science 334, 1141-1144 (2011).
9. Sutovsky, P. et al. Ubiquitinated Sperm Mitochondria, Selective Proteolysis, and the Regulation of Mitochondrial Inheritance in Mammalian Embryos. Biology of Reproduction 63, 582-590 (2000).
10. Parrish, J. et al. Mitochondrial endonuclease G is important for apoptosis in C. elegans. Nature 412, 90-94 (2001).
11. Li, L. Y., Luo, X. & Wang, X. Endonuclease G is an apoptotic DNase when released from mitochondria. Nature 412, 95-99. (2001).
12. Schwartz, M. & Vissing, J. Paternal Inheritance of Mitochondrial DNA. New England Journal of Medicine 347, 576-580 (2002).
13. Luo, S. et al. Biparental Inheritance of Mitochondrial DNA in Humans. Proc Natl Acad Sci USA 115, 13039-13044 (2018).
14. Pezzuto, J. M. Resveratrol: Twenty Years of Growth, Development and Controversy. Biomol Ther (Seoul) 27, 1-14 (2019).
15. Wang, Y. et al. Kinetics and specificity of paternal mitochondrial elimination in Caenorhabditis elegans. Nat Commun 7, 12569 (2016).
16. Ak, T. & Gulcin, I. Antioxidant and radical scavenging properties of curcumin. Chem Biol Interact 174, 27-37 (2008).
17. Alvares Delfino, V. D. et al. Folic acid therapy reduces plasma homocysteine levels and improves plasma antioxidant capacity in hemodialysis patients. Nutrition 23, 242-247 (2007).
18. Arrigoni, O. & De Tullio, M. C. Ascorbic acid: much more than just an antioxidant. Biochimica Et Biophysica Acta-General Subjects 1569, 1-9 (2002).
19. Boots, A. W., Haenen, G. R. & Bast, A. Health effects of quercetin: from antioxidant to nutraceutical. Eur J Pharmacol 585, 325-337 (2008).
20. Gill, B. S., Kumar, S. & Navgeet. Evaluating anti-oxidant potential of ganoderic acid A in STAT 3 pathway in prostate cancer. Mol Biol Rep 43, 1411-1422 (2016).
21. Gulcin, I. Antioxidant properties of resveratrol: A structure-activity insight. Innovative Food Science & Emerging Technologies 11, 210-218 (2010).
22. Gulcin, I., Huyut, Z., Elmastas, M. & Aboul-Enein, H. Y. Radical scavenging and antioxidant activity of tannic acid. Arabian Journal of Chemistry 3, 43-53 (2010).
23. Kang, K. A. et al. Baicalein inhibits oxidative stress-induced cellular damage via antioxidant effects. Toxicol Ind Health 28, 412-421 (2012).
24. Mari, M., Morales, A., Colell, A., Garcia-Ruiz, C. & Fernandez-Checa, J. C. Mitochondrial glutathione, a key survival antioxidant. Antioxid Redox Signal 11, 2685-2700 (2009).
25. Naguib, Y. M. Antioxidant activities of astaxanthin and related carotenoids. J Agric Food Chem 48, 1150-1154 (2000).
26. Singh, J. P., Selvendiran, K., Banu, S. M., Padmavathi, R. & Sakthisekaran, D. Protective role of Apigenin on the status of lipid peroxidation and antioxidant defense against hepatocarcinogenesis in Wistar albino rats. Phytomedicine 11, 309-314 (2004).
27. Valdez, L. B., Alvarez, S., Zaobornyj, T. & Boveris, A. Polyphenols and red wine as antioxidants against peroxynitrite and other oxidants. Biological Research 37, 279-286 (2004).
28. Lin, J. L. J. et al. Oxidative Stress Impairs Cell Death by Repressing the Nuclease Activity of Mitochondrial Endonuclease G. Cell Rep 16, 279-287 (2016).
29. Jia, K. & Levine, B. Autophagy and longevity: lessons from C. elegans. Adv Exp Med Biol 694, 47-60 (2010).
30. Salehi, B. et al. Resveratrol: A Double-Edged Sword in Health Benefits. Biomedicines 6 (2018).
31. Trela, B. C. & Waterhouse, A. L. Resveratrol: Isomeric molar absorptivities and stability. Journal of Agricultural and Food Chemistry 44, 1253-1257 (1996).
32. Nakagawa, A., Shi, Y., Kage-Nakadai, E., Mitani, S. & Xue, D. Caspase-dependent conversion of Dicer ribonuclease into a death-promoting deoxyribonuclease. Science 328, 327-334 (2010).
33. Buttner, S. et al. Endonuclease G mediates alpha-synuclein cytotoxicity during Parkinson's disease. EMBO J 32, 3041-3054 (2013).
34. Stefanis, L. alpha-Synuclein in Parkinson's disease. Cold Spring Harb Perspect Med 2, a009399 (2012).
35. Martinez, B. A. et al. Dysregulation of the Mitochondrial Unfolded Protein Response Induces Non-Apoptotic Dopaminergic Neurodegeneration in C. elegans Models of Parkinson's Disease. J Neurosci 37, 11085-11100 (2017).
36. McDermott-Roe, C. et al. Endonuclease G is a novel determinant of cardiac hypertrophy and mitochondrial function. Nature 478, 114-118 (2011).
37. Cote, J. & Ruiz-Carrillo, A. Primers for mitochondrial DNA replication generated by endonuclease G. Science 261, 765-769. (1993).
38. Wang, W. et al. Endonuclease G promotes autophagy by suppressing mTOR signaling and activating the DNA damage response. Nat Commun 12, 476 (2021).
39. DeLuca, S. Z. & O'Farrell, P. H. Barriers to male transmission of mitochondrial DNA in sperm development. Dev Cell 22, 660-668 (2012).
40. Vang, O. et al. What is new for an old molecule? Systematic review and recommendations on the use of resveratrol. PLoS One 6, e19881 (2011).
41. Shaito, A. et al. Potential Adverse Effects of Resveratrol: A Literature Review. Int J Mol Sci 21 (2020).
42. Bass, T. M., Weinkove, D., Houthoofd, K., Gems, D. & Partridge, L. Effects of resveratrol on lifespan in Drosophila melanogaster and Caenorhabditis elegans. Mech Ageing Dev 128, 546-552 (2007).
43. Kaeberlein, M. et al. Substrate-specific activation of sirtuins by resveratrol. J Biol Chem 280, 17038-17045 (2005).
44. Pacholec, M. et al. SRT1720, SRT2183, SRT1460, and resveratrol are not direct activators of SIRT1. J Biol Chem 285, 8340-8351 (2010).
45. Viswanathan, M., Kim, S. K., Berdichevsky, A. & Guarente, L. A role for SIR-2.1 regulation of ER stress response genes in determining C. elegans life span. Dev Cell 9, 605-615 (2005).
46. Wood, J. G. et al. Sirtuin activators mimic caloric restriction and delay ageing in metazoans. Nature 430, 686-689 (2004).
47. Lin, J. L., Chen, Y., Yang, W. Z., Chen, Y. N. & Yuan, H. S. Resveratrol stimulates mitochondrial Endonuclease G to degrade damaged DNA. accompanied submission (2022).
48. Britton, R. G., Kovoor, C. & Brown, K. Direct molecular targets of resveratrol: identifying key interactions to unlock complex mechanisms. Ann N Y Acad Sci 1348, 124-133 (2015).
49. Buttner, S. et al. Endonuclease G regulates budding yeast life and death. Mol Cell 25, 233-246 (2007).
50. Jang, M. et al. Cancer chemopreventive activity of resveratrol, a natural product derived from grapes. Science 275, 218-220 (1997).
51. Gu, T., Orita, S. & Han, M. Caenorhabditis elegans SUR-5, a novel but conserved protein, negatively regulates LET-60 Ras activity during vulval induction. Mol Cell Biol 18, 4556-4564 (1998).
52. Brenner, S. The genetics of Caenorhabditis elegans. Genetics 77, 71-94 (1974).
53. Wang, X., Yang, C., Chai, J., Shi, Y. & Xue, D. Mechanisms of AIF-mediated apoptotic DNA degradation in Caenorhabditis elegans. Science 298, 1587-1592 (2002).
54. Miller, D. M. & Shakes, D. C. Immunofluorescence microscopy. Methods Cell Biol 48, 365-394 (1995).
55. Jang, M. et al. Cancer chemopreventive activity of resveratrol, a natural product derived from grapes. Science 275, 218-220 (1997).
56. Pezzuto, J. M. Resveratrol: Twenty Years of Growth, Development and Controversy. Biomol Ther (Seoul) 27, 1-14 (2019).
57. Chaudhari, S. N. et al. Bacterial Folates Provide an Exogenous Signal for C. elegans Germline Stem Cell Proliferation. Dev Cell 38, 33-46 (2016).
58. Zhou, Q. et al. Mitochondrial endonuclease G mediates breakdown of paternal mitochondria upon fertilization. Science 353, 394-399 (2016).
59. Luo, S. et al. Biparental Inheritance of Mitochondrial DNA in Humans. Proc Natl Acad Sci USA 115, 13039-13044 (2018).
60. Schwartz, M. & Vissing, J. Paternal Inheritance of Mitochondrial DNA. New England Journal of Medicine 347, 576-580 (2002).

1. A compound according to Formula (IV) comprising: 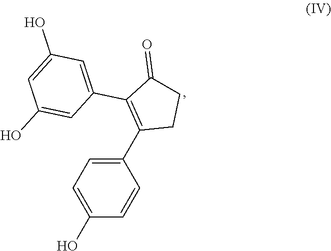 [SMILES: O=C1CCC(c2ccc(O)cc2)=C1c1cc(O)cc(O)c1]
or a pharmaceutically acceptable salt thereof.

2. A pharmaceutical composition comprising the compound of claim 1, and a pharmaceutically acceptable carrier.

3-10. (canceled)

11. A compound according to Formula (IV) comprising: 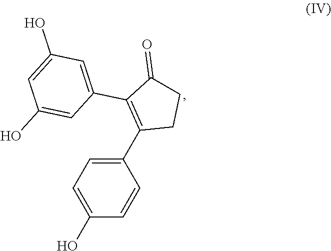 [SMILES: O=C1CCC(c2ccc(O)cc2)=C1c1cc(O)cc(O)c1]
or a pharmaceutically acceptable salt thereof, for use in a medicament for the treatment of an EndoG-related disease or condition, Parkinson's disease, neurodegeneration, and α-synuclein-induced neurodegeneration.

12-46. (canceled)

47. A therapeutic agent for the modulation of EndoG activity in a subject, comprising a mixture of trans- and cis-resveratrol isomers, and a pharmaceutically acceptable carrier, wherein the modulation of the nuclease activity of EndoG, or a homolog thereof is controlled by the relative quantity of trans- and cis-resveratrol isomers in said mixture wherein said trans- and cis-resveratrol isomers are stabilized trans- and cis-resveratrol isomers according to the compounds of Formula (IV) and (V) respectively: 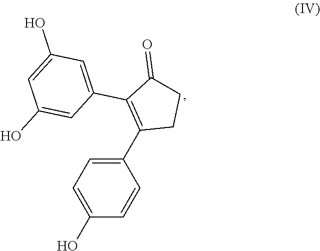 [SMILES: O=C1CCC(c2ccc(O)cc2)=C1c1cc(O)cc(O)c1]
and

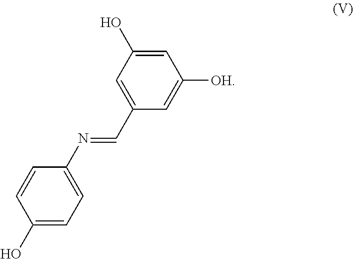 [SMILES: Oc1ccc(/N=C/c2cc(O)cc(O)c2)cc1]

48-59. (canceled)
